SEQUENCE LISTING (27 sequences)

SEQ ID NO: 1 - DNA, 20 bp, Artificial Sequence
  Primer
tgtggccagcgttttaccaa

SEQ ID NO: 2 - DNA, 20 bp, Artificial Sequence
  Primer
tgatgtgggctgtgcagttt

SEQ ID NO: 3 - DNA, 20 bp, Artificial Sequence
  Primer
aaaccccaccaagtaccaca

SEQ ID NO: 4 - DNA, 18 bp, Artificial Sequence
  Primer
acatggcaagctcaggac

SEQ ID NO: 5 - DNA, 20 bp, Artificial Sequence
  Primer
caagtaccagcccaggcggc

SEQ ID NO: 6 - DNA, 20 bp, Artificial Sequence
  Primer
gggtgccggtggtccaagtg

SEQ ID NO: 7 - DNA, 17 bp, Artificial Sequence
  Primer
gcgacgggctggaagag

SEQ ID NO: 8 - DNA, 20 bp, Artificial Sequence
  Primer
gctgtccgtgatggggtgcc

SEQ ID NO: 9 - DNA, 20 bp, Artificial Sequence
  Primer
ggaactggagcgtgcttttg

SEQ ID NO: 10 - DNA, 19 bp, Artificial Sequence
  Primer
ggcggttgctaaaccagac

SEQ ID NO: 11 - DNA, 19 bp, Artificial Sequence
  Primer
gaaagagcgagcgaggaga

SEQ ID NO: 12 - DNA, 20 bp, Artificial Sequence
  Primer
caggctgtagtggttagacg

SEQ ID NO: 13 - DNA, 21 bp, Artificial Sequence
  Primer
agatttgagatgctcatcccc

SEQ ID NO: 14 - DNA, 21 bp, Artificial Sequence
  Primer
gatgcgtgatgtcatactgga

SEQ ID NO: 15 - DNA, 22 bp, Artificial Sequence
  Primer
ccctagcctatatctccctttt

SEQ ID NO: 16 - DNA, 21 bp, Artificial Sequence
  Primer
taccatcgtggggataatggc

SEQ ID NO: 17 - DNA, 22 bp, Artificial Sequence
  Primer
gtcctccactcttttacagacg

SEQ ID NO: 18 - DNA, 21 bp, Artificial Sequence
  Primer
attcggttgtgaccaatgggt

SEQ ID NO: 19 - DNA, 20 bp, Artificial Sequence
  Primer
tgacggtggaccctgcccat

SEQ ID NO: 20 - DNA, 20 bp, Artificial Sequence
  Primer
agctttgcgtggcccgtagc

SEQ ID NO: 21 - DNA, 17 bp, Artificial Sequence
  Primer
gcagtcggaagtggcag

SEQ ID NO: 22 - DNA, 19 bp, Artificial Sequence
  Primer
caactcactgtcagatcaa

SEQ ID NO: 23 - DNA, 20 bp, Artificial Sequence
  Primer
cccagtgctggcctaatagc

SEQ ID NO: 24 - DNA, 22 bp, Artificial Sequence
  Primer
cacccttgatatccccaagtga

SEQ ID NO: 25 - DNA, 20 bp, Artificial Sequence
  Primer
caatccactgccatggcctt

SEQ ID NO: 26 - DNA, 25 bp, Artificial Sequence
  Primer
tcagataaagccaagtcgaatctgg

SEQ ID NO: 27 - DNA, 30 bp, Artificial Sequence
  Synthetic construct
gaaaaaaaagcatgacgtcaagccaggggg

(19) United States
(12) Patent Application Publication
(10) Pub. No.: US 2012/0095078 A1
(43) Pub. Date: Apr. 19, 2012

(54) METHODS OF DIAGNOSIS AND TREATMENT OF MELANOMA

(75) Inventor: Ze'ev Ronai, San Diego, CA (US)

(73) Assignee: Sanford-Burnham Medical Research Institute, La Jolla, CA (US)

(21) Appl. No.: 13/269,459

(22) Filed: Oct. 7, 2011

Related U.S. Application Data
(60) Provisional application No. 61/390,746, filed on Oct. 7, 2010.

(51) Int. Cl. A61K 31/713; C40B 30/04; A61P 35/00; C12Q 1/68

(52) U.S. Cl. 514/44 A; 435/6.14; 506/9; 435/6.12

(57) ABSTRACT

Methods for the diagnosis or prognosis of melanoma by detecting expression of ATF2 and MITF in melanocytes are provided herein. Also provided are methods of treating a melanocyte proliferative disorder with agents that modulate the transcriptional activity of ATF2 and/or MITF activity.

### CROSS REFERENCE TO RELATED APPLICATION(S)

This application claims the benefit of priority under 35 U.S.C. §119(e) of U.S. Ser. No. 60/390,746 filed Oct. 7, 2010 the entire content of which is incorporated herein by reference.

### BACKGROUND OF THE INVENTION

1. Field of the Invention

The invention relates generally to melanoma and more specifically to diagnosis and treatment of melanoma through microphthalmia-associated transcription factor (MITF) and activating transcription factor 2 (ATF2).

2. Background Information

Malignant melanoma is one of the most highly invasive and metastatic tumors and its incidence has been increasing at a higher rate than other cancers in recent years. Significant advances in understanding melanoma biology have been made over the past few years as a result of the identification of genetic changes along the MAPK signaling pathway. Those include mutations in BRAF, NRAS, KIT and GNAQ, all of which result in a constitutively active MAPK pathway. Consequently, corresponding transcription factor targets such as microphthalmia-associated transcription factor (MITF), AP2, and C-JUN and its heterodimeric partner ATF2 are activated and induce changes in cellular growth, motility and resistance to external stress. In addition, constitutively active MAPK/ERK causes rewiring of other signaling pathways. Among examples of rewired signaling is up-regulation of C-JUN expression and activity, which potentiates other pathways, including PDK1, AKT and PKC, and plays a critical role in melanoma development.

Activating transcription factor 2 (ATF2), a member of the bZIP family, is activated by stress kinases including JNK and p38 and is implicated in transcriptional regulation of immediate early genes regulating stress and DNA damage responses and expression of cell cycle control proteins. To activate transcription, ATF2 heterodimerizes with bZIP proteins, including C-JUN and CREB, both of which are constitutively up-regulated in melanomas. ATF2 is also implicated in the DNA damage response through phosphorylation by ATM/ATR. Knock-in mice expressing a form of ATF2 that cannot be phosphorylated by ATM are more susceptible to tumor development.

The transcription factor MITF, a master regulator of melanocyte development and biogenesis, has been shown to play a central role in melanocyte biology and in melanoma progression. Factors controlling MITF transcription have been well documented and include transcriptional activators, such as SOX10, CREB, PAX3, lymphoid enhancer-binding factor 1 (LEF1, also known as TCF), onecut domain 2 (ONECUT-2) and MITF itself, as well as factors that repress MITF transcription, including BRN2 and FOXD3. In addition, MITF is subject to several post translational modifications affecting its availability and activity, including acetylation, sumoylation and ubiquitination.

While previous studies indicate the presence of mutant BRAF in melanocytic lesions, as well as its effect on pigment gene expression, the role of MITF in early stages of melanoma development remains largely unexplored.

### SUMMARY OF THE INVENTION

The present invention is based on the seminal discovery of the role of ATF2 in de novo melanoma formation and the mechanism underlying ATF2's contribution to melanoma development. Transcriptionally active ATF2 is important for melanoma development and loss of transcriptionally active ATF2 allows higher expression of MITF. ATF2 negatively regulates MITF expression, and several other important pigmentation genes, in melanocytes. ATF2 regulation of MITF transcription in melanocytes is mediated by discrete promoter elements such as SOX10, JunB, FOXD3, and the like.

In one aspect, provided herein is a method for the diagnosis or prognosis of melanoma in a subject including obtaining a nucleic acid sample from melanocytes of a subject suspected of having melanoma or at risk of having melanoma; detecting expression of activating transcription factor 2 (ATF2) and microphthalmia-associated transcription factor (MITF) in melanocytes; and determining a ratio of ATF2:MITF expression, thereby allowing diagnosis or prognosis of melanoma in the subject. In one aspect, detection of expression is a comparison of nuclear and cytoplasmic localization of ATF2 expression in melanocytes. In another aspect, an increased ratio of nuclear localization of ATF2:MITF expression in melanocytes is associated with metastatic melanoma, is provided.

In another aspect, a method of treating a melanocyte proliferative disorder in a subject including administering to the subject an effective amount of an agent that modulates the transcriptional activity of ATF2, thereby treating the disorder, is provided. In certain aspects, the agent inhibits the transcriptional activity of ATF2. In one embodiment, the agent that modulates the transcriptional activity of ATF2 increases the expression of MITF. In another embodiment, the agent that modulates the transcriptional activity of ATF2 further modulates the transcriptional activity of SOX10 or further modulates the activity of melanocyte pigmentation genes. The pigmentation genes provided herein include, but are not limited to, Cyclin D1, ESAM1, Angiopoietin 2, Klflc, PCDH7, Silver, DCT, Tyrp1, and Tgfbi.

In one aspect, a method of treating a melanocyte proliferative disorder in a subject including administering to the subject an effective amount of an agent that modulates the transcriptional activity of ATF2 in combination with an anticancer drug, is provided. In one embodiment, the anticancer drug used for the treatment of melanoma, includes but is not limited to, vemurafenib (Zelboraf™).

The melanocyte proliferative disorder as provided herein includes melanoma, primary melanoma or metastatic melanoma.

### BRIEF DESCRIPTION OF THE DRAWINGS

FIG. 1. (A). schematically represents the targeting strategy and shows wild-type allele of Atf2 encompassing exons 8 and 9 (boxes) and flanking loxP sequences (arrowheads). (B). is a photographic representation of ATF2 expression assessed by immunoblot in melanocytes derived from TyrCre⁺::Aft2^{+/+}::Nras^{Q61K}::lnk4a^{-/-} and TyrCre⁺::Atf2^{md}::Nras^{Q61K}::lnk4a^{-/-} mice and treated with 4-OHT, enabling expression of a mutant lacking the DNA binding and leucine zipper domains where P-actin served as a loading control. (C). photographically depicts induction of mutant NRAS in melanocytes. Shown is RT-PCR analysis (20 cycles) of NRAS^{Q61K} transcript levels in melanocyte cultures treated with doxycycline (2 mg/ml). RNA from untreated melanocytes served as a control (right lane), and Cyclophilin A served as an internal control. (D). is a photographic representation of melanoma development in TyrCre⁺::Atf2^{md}::Nras^{Q61K}::lnk4a^{-/-} mice. Image represents animals of TyrCre⁺::Atf2^{+/+}::Nras^{Q61K}::lnk4a^{-/-} or TyrCre⁺::Atf2^{md}::Nras^{Q61K}::lnk4a^{-/-} genotype that were analyzed for tumor formation within 8-32 weeks.

FIG. 2 is a photograph showing slides of melanoma tumors stains from mice of the TyrCre⁺: :Atf2^{+/+}Nras^{Q61K}lnk4a^{-/-} genotype for melanoma markers. Immunohistochemistry was performed on 5 μM paraffin-embedded samples. Sections were incubated with S-100 and DCT antibody (Ab) or control secondary antibody (Cont) and counterstained with hematoxylin. Scale bar=50 micron. Slides were scanned by scanscope at 20× (IHC) and 40× (H&E).

FIG. 3. (A). is a graphical representation of qPCR analysis performed on RNA prepared from skin of 4-day-old ATF2 WT or Atf2^{md} mice (3 mice per sample). (C). graphically depicts quantification of immunostaining performed using the automated Aperio ScanScope CS system. The percentage (%) reflects the amount of positive signal in five selected fields representing longitudinal sections through the skin and containing the entire length of hairs, from the bulb with the subcutis to the epidermis.

FIG. 4 is a photograph of slides representing IHC analysis of mouse skin prepared from 4-day-old mice. Magnifications shown are 20 and 40×. S100 staining reveals changes in the hair matrix (low magnification) and migrating melanoblasts (higher magnification). Scale bar=100 micron.

FIG. 5. (A). is a photograph (upper panel) of a gel that shows ATF2 knock down in primary human melanocytes (one 10 cm plate each, 50% confluent) where cells were either untreated or kept under hypoxia (1%) for 16 hours. Cells were lysed and Western blotting was carried out with the indicated antibodies. The lower panel is a graphic representation of RNA extracted from the above samples with qPCR carried out using MITF primers. Cyclophilin A served as an internal control. (B). is a photograph (upper panel) of a gel showing ATF2 knock down in melan-lnk4a-Arfl mouse melanocytes with Western blotting performed using the indicated antibodies. The lower panel (B) graphically depicts qPCR analysis of the above samples carried out using MITF primers.

FIG. 6(C). Left panel is a photograph of a gel that shows Lul205 melanoma cells (one 10 cm plate each, 50% confluent) transduced with either empty or shATF2 lentiviral vectors. Cells were left untreated, kept under hypoxia (1%), or treated with UV-B (21 mJ/cm² for 6 hours) irradiation. Proteins were prepared 6 hours after UV-B treatment or 16 hours after growth under hypoxia. Proteins were immunoblotted using the indicated antibodies. (C) right pane is a bar graph representation that shows RNA extracted from cells maintained under the same conditions with qPCR performed using MITF primers. Cyclophilin A served as an internal control for qPCR. (D). depicts the experiment described in (C), which was duplicated using MeWo melanoma cells.

FIG. 7 is a graphic representation showing ATF2 knock down in human melanocytes (Hermes 3A). qPCR was carried out using primers for the indicated mRNAs.

FIG. 8. (B). is a bar graph representation of relative fold change in melanoma cells (WM1361, one 10 cm plate each, 50% confluent) transduced with either empty or shATF2 lentiviral particles. Cells were selected for 3 days with puromycin treatment (1.5 μg/ml) and then transfected with either a WT MITF-luciferase reporter or one with a mutant BRN2 site (0.5 μg) along with P-gal (0.1 μg) as an internal control. P-gal activity was normalized for every sample and the relative fold change in reporter activity for control and shATF2 cells is shown. The right panel is a bar graph depicting relative fold change performed in WM1361 melanoma cells using a WT or CRE-mutated MITF promoter. (C). is a bar graph representation of the analysis performed as in (B) but using human melanocytes H3A (left panel) or 1361 melanoma cells (right panel) and the WT MITF promoter or a construct in which the SOX10 site was mutated. (D). is a photograph of a gel showing human melanocytes (H3A); one 10 cm plate each, 50% confluent) infected with control shRNA (SiSC) or shATF2. After puromycin selection for 3 days, control shRNA cells and ATF2 knock down cells (2×10⁶) were transfected with either scrambled siRNA (SiSC) or siRNA against SOX10. After 72 hours, Western analysis was carried out using 50 μg of proteins and the indicated antibodies. (E). is a photograph of a gel depicting the same analysis described in (D) but performed in human melanoma cells. The right panel is a graphic representation of RNA extracted from cells with qPCR analysis of MITF transcripts.

FIG. 9(A). is a photographic representation of a gel of chromatin immuno-precipitation performed using antibodies to SOX10 or CREB (or rabbit IgG as control) in WM1361 melanoma cells expressing either control shRNA or ATF2 shRNA. The MITF promoter sequence spanning −300 to −120 (180 bp containing SOX10 binding sites and a CRE site) was amplified using MITF-specific primers. GAPDH served as a control. (B). photographically depicts a gel of ChIP performed using the indicated antibodies followed by amplification of the SOX10 promoter sequence containing AP-1 binding sites (−4797 to −4791). GAPDH served as control.

FIG. 10. (C). is a photograph of a gel depicting ChIP analysis carried out using ATF2 or JunB antibodies in cells that express either shcontrol, shATF2 or Jun DN construct TAM67. (D). photographically depicts a gel indicating expression of JunB or JunD (3 μg) alone or in combination with TAM67 (2 μg) performed in WM1361 melanoma cells where expression of SOX10 protein was monitored in westerns and quantified using the LICOR imaging system. The lower panel, is a graphic representation of corresponding changes in level of MITF transcripts assessed by qPCR. (E). photographically (left panel) and graphically depicts (right panel) the experiment described in (D) but replicated with the melanocytes H3A cell line. (F). is a photograph of a gel (left panel) that shows the effect of JunB on SOX10 expression assessed in melanoma WM1361 cells expressing control or shATF2 and a bar graph (right panel), which shows the level of MITF transcripts quantified by qPCR.

FIG. 11(A). is a photograph of a gel showing Melan-lnk4a-Arfl cells stably transduced with wild type BRAF or BRAF^{V600E}, followed by treatment with ICI 182780 (ICI, 200 nM) to induce BRAF expression. Western analysis was carried out to assess ERK activation using pERK and ERK antibodies. (B). is a bar graph that indicates quantification of mouse melanocytes stably expressing mutant B-RAF that were infected with lentiviral vectors carrying shRNA for ATF2 or MITF or both. Cells were plated (5000 per well) on soft agar and assessed for the ability to form colonies after 21 days using P-Iodonitrotetrazolium Violet staining. Colonies were counted in triplicate wells per experiment, and experiments were reproduced twice. Means+SD are shown. (C). shows representative photographic images of colonies for the quantification depicted in (B). (D). is a series of bar graphs depicting qPCR performed for MITF (left panel) and ATF2 (middle panel) transcripts using RNA from cells used in (B) and (C). The right panel of (D) is a photograph of a gel showing western analysis performed on lysates obtained from cells used for colony formation with the indicated antibodies.

FIG. 12(E). is a bar graph depicting mouse melanocytes analyzed for colony formation stained with BrdU relative to a control. The cell cycle phase was analyzed using the Mod Fit LT v.2 program. The percentage of cells in G1, S and G2 is shown in the graph. (F). shows scatter plots for melanocytes described in FIG. 11 and FIG. 12(E) that were also stained with Annexin V (early apoptosis) and 7-AAD (apoptosis and necrosis). The plots presented reveal Annexin-APC staining on the x-axis and 7-AAD staining on the y-axis. Mean+/−SD are calculated based on triplicate analyses.

FIG. 13 graphically depicts the analysis of nuclear ATF2 and MITF in melanoma specimens using quantitative immunofluorescence. ATF2/MITF expression ratios for metastatic and primary specimens are shown.

FIG. 14 is a graphic representation of nuclear ATF2 and MITF in melanoma specimens using quantitative immunofluorescence. ATF2/MITF ratios associated with decreased 10-year disease specific survival are shown as well as the number of cases and events (deaths) for high or low ATF2/MITF expression ratios. Quantification was performed by AQUA, which provides continuous output scores. In the absence of underlying cut-point justification, nuclear ATF2/MITF ratios were randomly binarized by the median ratio for all specimens (primary and metastatic)

FIG. 15 is a table showing the tumor incidence in mice with mixed genetic backgrounds.

FIG. 16 is a table showing tumors that were positive for melanoma markers S100 and DCT.

FIG. 17 is a table with results from microarray analysis of ATF2^{+/+}TyrCre⁺Nras⁺Ink4a^{-/-} and ATF2^{-/-}TyrCre⁺Nras⁺Ink4a^{-/-} melanocytes and confirmation by qPCR.

FIG. 18 is a table depicting MITF and SOX10 mRNA levels in melanocytes and melanoma cell lines following ATF2 knock down (KD).

### DETAILED DESCRIPTION OF THE INVENTION

The present invention is based on the association between ATF2 and melanoma development and the diagnosis and prognosis of patients relating to melanocyte transformation and melanoma, including metastasis development.

To directly assess the importance of ATF2 in melanoma development, a mouse melanoma model in which ATF2 is selectively inactivated in melanocytes was employed. Melanoma development was demonstrated to be markedly attenuated in mice expressing a transcriptionally inactive form of ATF2 in melanocytes. Surprisingly, ATF2 control of melanoma development was mediated, in part, through its negative regulation of SOX 10 and consequently of MITF transcription. Inhibition of ATF2 abolished mutant BRAF-expressing melanocytes' ability to form foci on soft agar, which was partially rescued when expression of MITF was attenuated. The significance of these findings is underscored by the observation that human melanoma tumors having a high ratio of nuclear ATF2 to MITF expression were associated with poor prognosis. Thus, a novel mechanism underlying melanocyte transformation and melanoma development has been identified.

The transcription factor ATF2 has been shown to attenuate melanoma susceptibility to apoptosis and to promote its ability to form tumors in xenograft models. The role of ATF2 in melanoma development has been demonstrated by analysis of a mouse melanoma model crossed (Nras^{Q61K}::lnk4a^{-/-}) with mice expressing a transcriptionally inactive form of ATF2 in melanocytes.

In contrast to 7 of 21 of the Nras^{Q61K}::lnk4a^{-/-} mice, only 1 of 21 mice expressing mutant ATF2 in melanocytes developed melanoma. Gene expression profiling identified higher MITF expression in primary melanocytes expressing transcriptionally inactive ATF2. MITF downregulation by ATF2 was confirmed in the skin of Atf2^{-/-} mice, in primary human melanocytes and in 50% of the human melanoma cell lines. Inhibition of MITF transcription by MITF was shown to be mediated by ATF2-JunB-dependent suppression of SOX10 transcription. Remarkably, oncogenic BRAF (V600E)-dependent focus formation of melanocytes on soft agar was inhibited by ATF2 knockdown and partially rescued upon shMITF co-expression. On melanoma tissue microarrays, a high nuclear ATF2 to MITF ratio in primary specimens was associated with metastatic disease and poor prognosis. These findings establish the importance of transcriptionally active ATF2 in melanoma development through fine-tuning of MITF expression.

Negatively regulation of MITF transcription by the transcription factor ATF2 in melanocytes and in about 50% of melanoma cell lines has been demonstrated herein. Increased MITF expression, which was observed upon inhibition of ATF2, effectively attenuated the ability of BRAF^{V600E}-expressing melanocytes to exhibit a transformed phenotype, an effect partially rescued when MITF expression was also blocked. Significantly, the development of melanoma in mice carrying genetic changes observed in human tumors was inhibited upon inactivation of ATF2 in melanocytes. Melanocytes from mice lacking active ATF2 expressed increased levels of MITF, confirming that ATF2 negatively regulates MITF and implicating this newly discovered regulatory link in melanoma development. Primary melanoma specimens that exhibit a high nuclear ATF2 to MITF ratio were found to be associated with metastatic disease and poor prognosis, further substantiating the significance of MITF control by ATF2. In all, these findings provide genetic evidence for the role of ATF2 in melanoma development and indicate an ATF2 function in fine-tuning MITF expression, which is central to understanding MITF control at the early phases of melanocyte transformation.

The loss of a transcriptionally active form of ATF2 in melanocytes inhibits melanoma development in an Nras/lnk4a model. A critical role for ATF2 regulation of MITF, an important regulator of melanocyte biogenesis and a factor implicated in melanoma progression, has been identified during the course of an analysis of the mechanisms underlying ATF2 activity in this process. Surprisingly, ATF2 has been found to negatively regulate MITF expression in mouse and human melanocytes, suggesting that ATF2 transcriptional activities limit MITF expression. As demonstrated herein, such negative regulation is elicited through downregulation of SOX10 by ATF2, in cooperation with JunB. A putative API response element has been identified in SOX10 promoter sequences and ChIP analysis of this domain showed ATF2 and JunB binding. Over-expression of JunB efficiently suppressed SOX10 expression in an ATF2-dependent manner and inhibition of Jun transcriptional activities phenocopied the effect of shATF2, suggesting that negative regulation of SOX10 by ATF2 is direct, and is mediated in cooperation with JunB.

Importantly, ATF2-dependent negative regulation of Sox 10 and consequently of MITF is seen in melanocytes, but only in about 50% of the 18 melanoma cell lines studied here. Correspondingly, JunB, which is required for ATF2-dependent inhibition of Sox10 transcription, is no longer found on the promoter of SOX10 in melanoma cells (i.e., 501Mel) that exhibit positive regulation by ATF2. Rather, CREB and ATF2 are found on SOX10 and MITF promoters, pointing to a switch in ATF2 heterodimeric partners to enable positive regulation of these genes. Notably, melanoma cell lines that exhibit positive regulation of SOX10 and MITF by ATF2, also show high basal levels of MITF expression, suggesting that additional genetic or epigenetic changes distinguish these lines from melanocytes and the other melanoma lines in which ATF2 elicits negative regulation of MITF.

ATF2 control of MITF expression affected the ability of BRAF^{600E}-expressing melanocytes to exhibit transformed phenotype in culture, monitored by their ability to grow on soft agar. Inhibition of ATF2 abolished soft agar growth of BRAF^{600E}-expressing melanocytes, which was partially rescued upon knock down (KD) of MITF. Interestingly, both the over-expression or the KD of MITF resulted in inhibition of melanocytes ability to grow on soft agar, substantiating the notion that a fine balance of MITF expression must be maintained in order to ensure its contribution to cellular proliferation and transformation. Thus, while excessively low or high MITF levels appear to block melanocyte transformation, intermediate levels allow transformation to occur. Clearly ATF2 plays an important role in fine-tuning MITF levels, which is consistent with the rheostat model proposed for MITF's role in melanoma development and progression.

Of importance, ATF2 and MITF affect the ability of BRAF^{600E}-expressing melanocytes to grow on soft agar via distinct mechanisms. Whereas specific inhibition of ATF2 causes both accumulation of cells in G2 and induction of cell death, specific alteration of MITF protein levels, particularly depletion, significantly affects cell proliferation and inhibit growth on soft agar by non-lethally slowing cell cycle progression at G2/M. These observations are consistent with a report from Wellbrock and Marais, who showed that altered MITF expression inhibits melanocyte proliferation.

Importantly, inhibiting MITF expression in ATF2 KD melanocytes was sufficient to partially rescue melanocyte growth on soft agar. While supportive of findings in the Nras::lnk4a mouse melanoma model, where expression of transcriptionally inactive ATF2 inhibits melanoma formation, these observations provide the foundation for a model in which ATF2 inhibition causes increased MITF levels and concomitant inhibition of melanocyte growth, possible induction of cell death and delayed development. The latter is suggested by IHC analysis of mouse skin from ATF2^{md} mice, which shows notably reduced S100 staining indicative of delayed melanocyte development: ATF2 KO melanocytes appear to represent anagen stage IV, whereas WT represent anagen stage VI. This delay was seen at the 4- but not the 14-day time point, suggesting that an ATF2 effect might be limited to a specific subpopulation or phase of melanocyte development. The early (4 day) time point is within the time frame that allows induction of melanoma development by UV-irradiation of c-Met or H-Ras mutant mice. It is therefore plausible that timely control of MITF expression by ATF2 determines melanocyte susceptibility to transformation.

An analysis of genes whose expression is altered by ATF2 KD in melanocytes identified a cluster of pigmentation genes, many reportedly regulated by MITF. Therefore, changes in TYRP1, DCT and SILVER expression could be attributed to altered MITF expression. However, an initial analysis points to a more complex mechanism since (i) the degree of changes in expression of these genes was often greater than that seen for MITF; and (ii) expression of some pigmentation genes was found to be independent of MITF in some melanoma and melanocyte cultures. Hence, further studies are required to address mechanisms underlying ATF2 regulation of these pigmentation genes and the significance of such regulation to melanocyte transformation and melanoma development. While present studies focused on the ATF2-MITF axis, it is expected that additional ATF2-regulated genes contribute to melanoma development. In agreement, previous studies using both human and mouse melanoma lines demonstrate that inhibition of ATF2 effectively inhibits tumorigenesis and blocks metastasis.

Important for ATF2 function is its subcellular localization. Nuclear localization of ATF2 in melanoma tumor cells is associated with poor prognosis, likely due to transcriptional activity of constitutively active ATF2. Indeed, expression of transcriptionally inactive ATF2 or peptides that attenuate endogenous ATF2 activity inhibits melanoma development and progression in xenograft models. These studies suggest that ATF2 is required for melanoma development and progression.

While the findings disclosed herein position ATF2 as an oncogene functioning in melanocyte transformation and melanoma development, earlier studies suggest that in keratinocytes and mammary glands, ATF2 elicits a tumor suppressor function. An assessment of the localization of ATF2 in the melanoma cell lines studied here has revealed that all express nuclear ATF2. Interestingly, in most cases the nuclear staining revealed a punctate staining, resembling the localization of ATF2 to DNA repair foci following DNA damage. A possible link between the presence of ATF2 in repair foci in most melanoma cells points to the possible presence of activated DNA damage response which may be associated with genomic instability, aspects that will be explored in detail in future studies. Significantly, the appearance of nuclear ATF2 is correlated with poor prognosis in melanoma, whereas melanomas that exhibit cytosolic ATF2 exhibit a better survival. Cytosolic ATF2 is reported to be primarily seen in non-malignant skin tumors. As demonstrated herein, high nuclear ATF2/MITF ratios are associated with poor prognosis in primary melanomas, but not with metastatic melanomas. The latter finding attests for the important role ATF2 plays to control M1TF expression in the early phase of melanocyte transformation and melanoma development.

Overall, using the mutant Nras/lnk4a melanoma model, genetic evidence for a central role for ATF2 in melanoma development is provided herein. In the absence of transcriptionally active ATF2, melanoma formation is largely inhibited. Furthermore, the data presented herein point to an unexpected role of ATF2 in fine-tuning of MITF transcription through regulation of its positive regulator SOX10. Mouse melanoma models and in vitro transformation studies indicate that this newly identified regulatory pathway is required for early phases of melanocyte transformation. Given that ATF2 affects activity of the oncogenes N-Ras (mouse model) and BRAF (melanocyte growth on soft agar), it is likely that ATF2 plays significant roles in melanomas that carry either of these mutations.

The following examples are intended to illustrate but not limit the invention.

### EXAMPLE 1

### Generation of Melanocyte-Specific ATF2 Mutant Mice

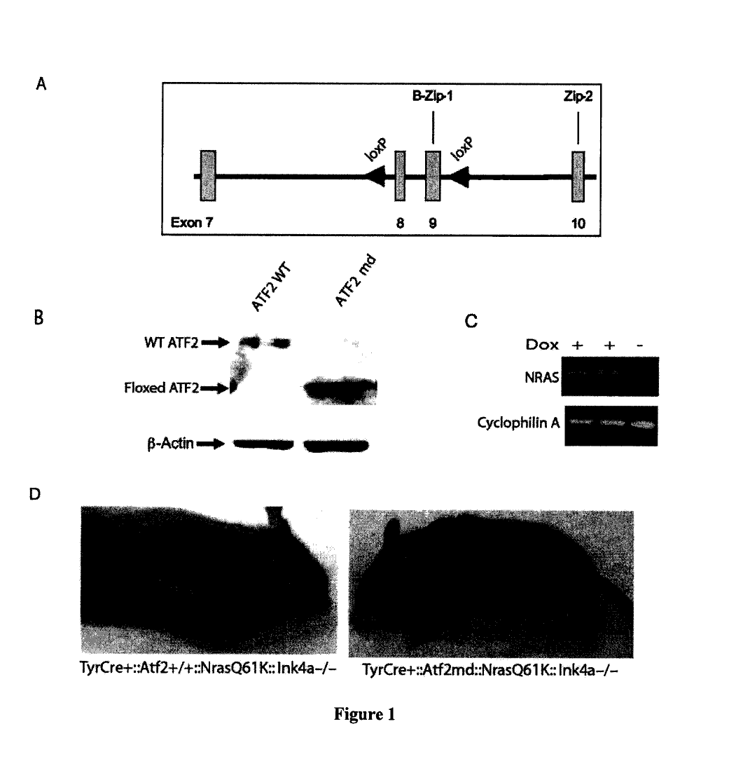

Because global Atf2 knockout in mice leads to early post-natal death, the Cre-loxP system was utilized to disrupt Atf2 in melanocytes. Deletion of its DNA binding domain and a portion of the leucine zipper motif results in a transcriptionally inactive form of ATF2 (FIG. 1A). To generate loss-of-function mutants, mice that would allow CRE-dependent deletion of these domains were established. Mice homozygous for the loxP-fianked (floxed) Atf2 gene (Atf2^{f/f}) were born at the expected Mendelian ratios and presented no apparent abnormalities. In addition, in several tissues analyzed, ATF2 expression levels were comparable between WT and Atf2^{f/f} mice (data not shown).

To elucidate the role of ATF2 in melanoma, Atf2^{f/f} mice were crossed with mice harboring a 4-hydroxytamoxifen (OHT)-inducible Cre recombinase-estrogen receptor fusion transgene under the control of the melanocyte-specific tyrosinase promoter, designated Tyr::Cre^{ER} (T2). It was anticipated that, upon administration of OHT, CRE-mediated recombination would be induced in a spatially and temporally controlled manner in embryonic melanoblasts, melanocytes, and in putative melanocyte stem cells. The resulting Atf2^{f/f}/Tyr-Cre^{ER} (T2) mice, designated melanocyte-deleted (md) Atf2^{md}, expressed the gene encoding the ATF2 transcriptional mutant in melanocytes as predicted. Immunoblot analysis of ATF2 protein confirmed that melanocytes prepared from wild-type TyrCre⁺::Atf2^{+/+}Nras⁺::lnk4a^{-/-} (WT) mice express a 70 kDa band corresponding to full length ATF2, whereas melanocytes of TyrCre⁺::Atf2^{md}::Nras⁺::lnk4a^{-/-} mice express only a 55 KDa band, corresponding to the size of ATF2 lacking the DNA binding and leucine zipper domains (FIG. 1B).

### EXAMPLE 2

### Disruption of ATF2 in Melanocytes Inhibits Melanoma Formation

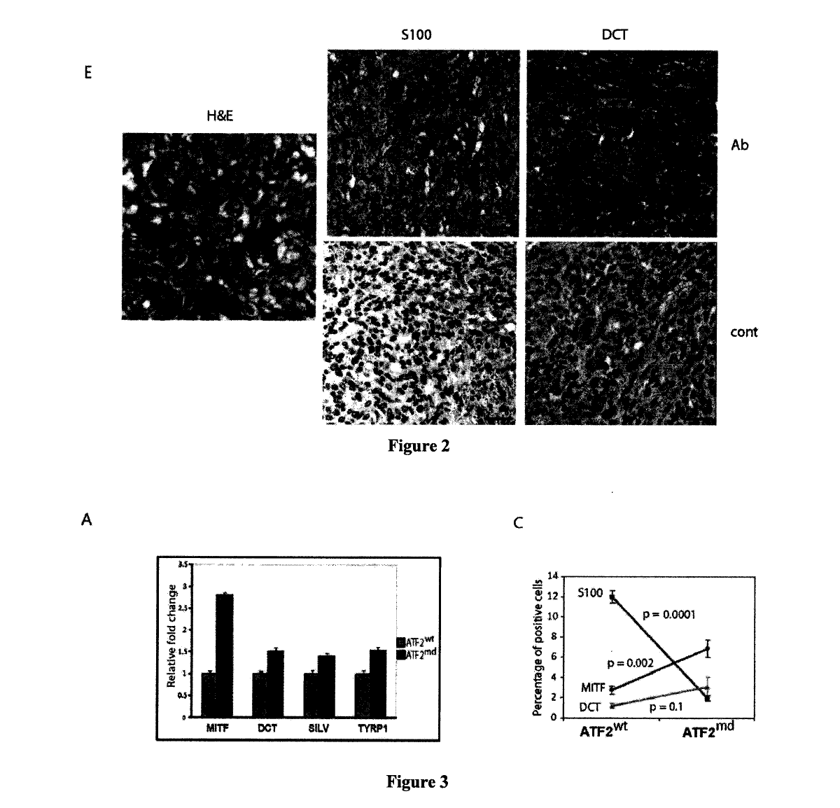

To address the role of ATF2 in de novo melanoma formation, Tyr: :Cre^{ER}: :Nras^{Q61K}: :lnk4a^{-/-} (KO of exon 2-3 of Cdkn2a locus that encodes for both p16^{lnk4a} and p19^{Arf}) mice, which develop spontaneous melanoma (Lynda Chin, unpublished observations), were crossed with Atf2^{md} mice. Similar to findings reported by Ackermann et al. (Cancer Res., 2005, 65: 4005-4011), mutant N-Ras/lnk4a^{-/-} mice developed melanoma within 8-12 weeks with metastatic lesions often seen in the lymph nodes. However, the incidence of melanoma was lower in Tyr::Cre^{ER}::Nras^{Q61K}::lnk4a^{-/-} mice used in the present study (50% penetrance, of which 50% of the tumors were confirmed to be melanoma) and this was attributed to expression of mutant NRAS induced only after birth, as opposed to activation of NRAS during embryogenesis, as reported by Ackermann et al. Thus, Atf2^{md}::N-Ras^{Q61K}:lnk4a^{-/-} mice were used to assess changes in melanoma incidence in the absence of functional ATF2 over a period of up to 8 months, in all cases, mouse skin was treated with Tamoxifen within 3-5 days after birth to inactivate ATF2 (FIG. 1B) and with doxycycline in their drinking water to induce expression of the NRAS mutant transgene (See Example 10 for details; FIG. 1C). In the control group (Tyr::Cre^{ER}::Atf2^{+/+}::Nras^{Q61K}::lnk4 a^{-/-}), 11/21 mice (52%) developed tumors within 8-16 weeks (see Table 1). In ATF2 heterozygotes (Tyr::Cre^{ER}::Atf2^{-/+}::Nras^{Q61K}::lnk4a^{-/-}), 18/44 mice (41%) developed tumors within 8-16 weeks, and in the Tyr::Cre^{ER}:Atf2^{md} :Nras^{Q61K}::lnk4a^{-/-} group only 3 of 21 animals (15%) developed tumors within 24-36 weeks (FIG. 1d and Table 1). To evaluate tumor type, melanoma markers including DCT and S100 were examined in all tumors (FIG. 2). This analysis identified 55-63% of tumors as melanomas in both the Atf2^{+/+} (7/11) and Atf2^{+/-} (10/18) groups (Table 2). Only one of the three tumors observed in the Atf2^{md} group was identified as a melanoma. Kaplan Meier curve did not reveal significant differences in survival among the different genotypes and this was ascribed to the study design, which was devised primarily to follow tumor incidence. Common to all genotypes, most tumors that were not identified as melanomas were fibrosarcomas and lymphomas, consistent with previous reports by Lynda Chin. These data suggest that transcriptionally active ATF2 is required for melanoma development in the Nras^{Q61K}::lnk4a^{-/-} mouse melanoma model.

### EXAMPLE 3

### Identification of MITF as an ATF2-Regulated Gene

To assess the mechanism underlying ATF2′s contribution to melanoma development, gene profiling array analysis of primary melanocytes prepared from Tyr: :Cre⁺::Atf2^{+/+}::Nras^{Q61K}::lnk4a^{-/-} and Tyr::Cre⁺::Atf2^{md}::Nras^{Q61K}::lnk4a^{-/-} mice was conducted. Because, as reported above, only one melanoma formed in the ATF2 mutant group, the analysis was limited to melanocytes. In all cases, ATF2 was inactivated and NRAS was induced in culture within 48 hours of plating cells, as monitored by western blots (FIG. 1b, 1c, and data not shown). Melanocytes were enriched, and immunostaining with appropriate markers confirmed that samples were free of keratinocytes and fibroblasts (data not shown; see Example 10 for details). RNA was prepared from cultures and two biological and technical replicates were used for data analysis. As shown in Table 3, among transcripts differentially expressed in ATF2 WT and mutant cultures were several factors that play an important role in melanocyte pigmentation, including Mitf, Silver, Tyrpl and Dct.

qPCR analysis, performed on independently prepared RNA samples from melanocytes expressing WT (Tyr:Cre⁺::Atf2^{+/+}::Nras^{Q61K}::lnk4a^{-/-}) or mutant ATF2 (Tyr::Cre⁺::Atf2^{md}::Nras^{Q61K}::lnk4a^{-/-}), confirmed altered expression of pigmentation genes (Table 3). These data provide the initial indication that ATF2 negatively regulates Mitf and several other important pigmentation genes. The pigmentation genes identified in this array are already known to be regulated by MITF thus, regulation of MITF by ATF2 was investigated in further detail.

### EXAMPLE 4

### MITF is Negatively Regulated by ATF2 in Mouse and Human Melanocytes

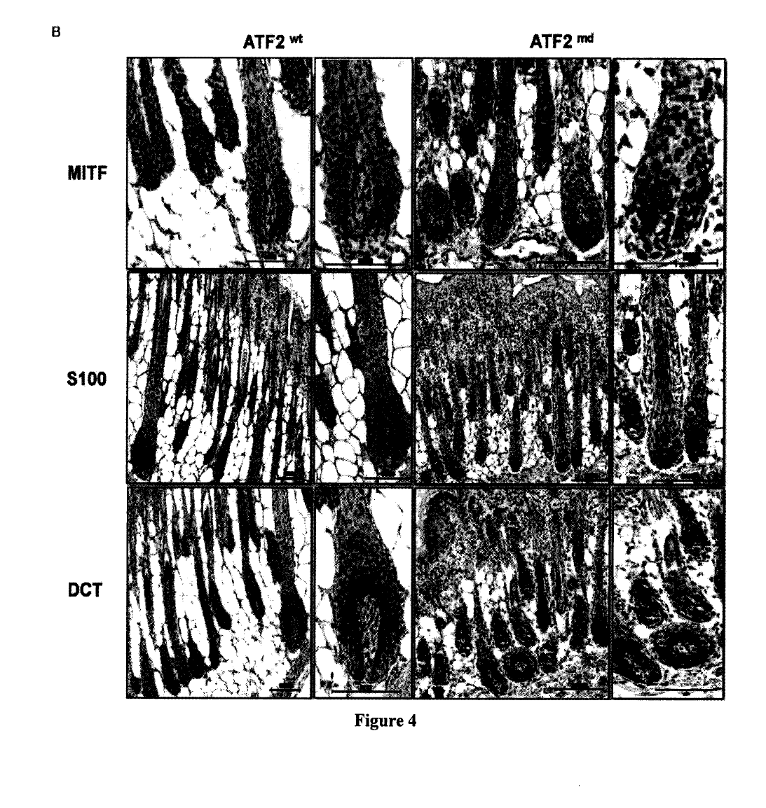

To confirm that ATF2 negatively regulates Mitf expression, MITF transcription in primary mouse melanocytes harboring WT (Tyr::Cre::Atf2^{+/+}) or mutant (Tyr::Cre⁺::Atf2^{md}) forms of ATF2 was assessed. RNA prepared from whole skin of these mice (3 mice per group) was subjected qPCR analysis. Significantly, Mitf expression was inversely correlated to the presence of functional ATF2; samples obtained from ATF2 mutant skin exhibited a greater than 2-fold increase in MITF expression compared with those obtained from WT ATF2 mice (FIG. 3A). Additionally, genes transcriptionally regulated by MITF, such as Dct, Silver and Tyrp1, were found to be upregulated in the skin of mutant ATF2 mice (FIG. 3A). The degree of altered expression of pigmentation genes was less pronounced in whole skin samples than in cultured melanocytes (Table 3), probably due to confounding effects of in vitro cell culture. To confirm the qPCR data, immunostaining of skin tissue samples obtained from 4 days old WT or ATF2 mutant mice was performed and increased MITF expression in melanocytes from Atf2^{md} mice relative to their WT counterparts (FIG. 4) was observed. Quantification of MITF staining revealed an approximate 2-fold increase in nuclear MITF expression in Atf2^{md} compared to WT mice (FIG. 3C). Notably, the level of 5100 staining in the hair matrix was markedly reduced in the skin of Atf2^{md} mice. At a later time point (2 weeks) representing an advanced stage of melanocyte development, S100 staining was similar in both genotypes, while MITF expression remained upregulated in Atf2^{md} mice (not shown). These data confirm initial observations in primary mouse melanocytes that MITF levels are elevated in ATF2 mutant-expressing cells.

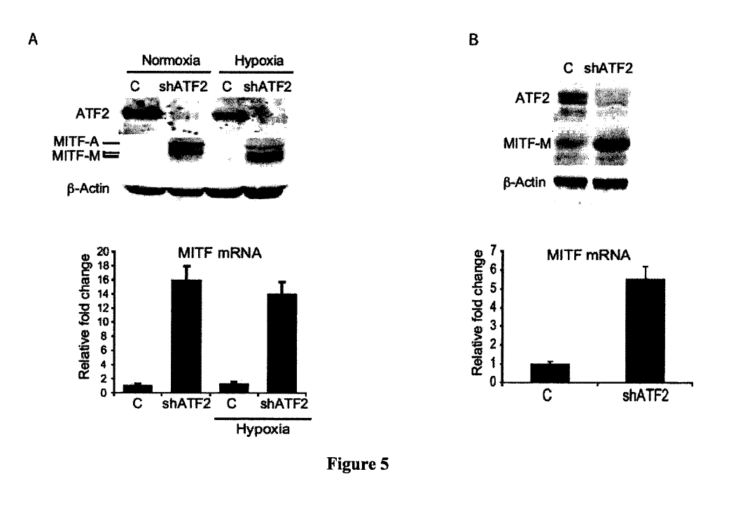

Additionally, melan-lnk4a-Arf1 melanocytes, a line derived from black Ink4a-Arf null mice, and primary human melanocytes were assessed. In both, ATF2 expression was inhibited by viral infection with the corresponding mouse or human shRNA (shATF2). Infection of either primary human (FIG. 5A) or melan-lnk4a-Arf1 melanocytes (FIG. 5B) with shATF2 markedly increased MITF transcription and protein expression (FIGS. 5A and 5B). These findings show that loss of transcriptionally active ATF2 allows higher expression of MITF and is strongly suggestive that ATF2 negatively regulates MITF expression in melanocytes.

### EXAMPLE 6

### MITF Transcription is Negatively Regulated by ATF2 in About 50% of Human Melanoma Cells

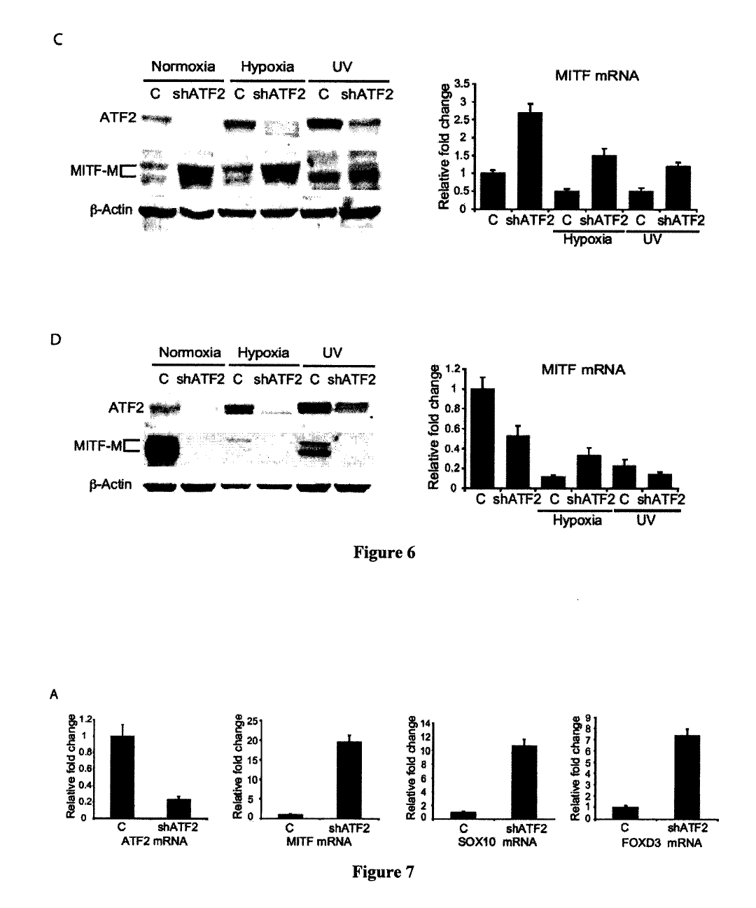

Given that ATF2 negatively regulates MITF in melanocytes of mouse and human tissues and in related melanocyte cell lines, the role of ATF2 in the regulation of MITF in human melanoma cells was investigated. Initially, changes in MITF expression was assessed in six human melanoma lines harboring oncogenic mutations in BRAF or NRAS in which ATF2 expression was effectively inhibited by corresponding shRNA (shATF2). In all cases, shRNA specificity was confirmed using three independent sequences (data not shown). Surprisingly, the six melanoma lines fell into two classes based on distinct patterns of regulation of MITF by ATF2 (Table 4). The first class comprised four of the six melanoma cultures (1205Lu, WM35, WM793 and WM1361), in which MITF expression was elevated 3-6-fold following inhibition of ATF2 expression (FIG. 6C). Conversely, a second class of cells, including MeWo and 501Mel cells, exhibited decreased MITF expression after ATF2 knockdown (KD), suggesting positive regulation of MITF by ATF2 (FIG. 6D). Notably, this latter group showed high levels of basal MITF expression, suggesting that regulation of MITF expression in these cells differs mechanistically from that of the first group. Further, in response to stress (UV or hypoxia) the MeWo and 501Mel lines further reduced MITF expression (FIG. 6D and data not shown), providing further evidence for differential regulation of MITF in these cells both prior to and in response to stress stimuli. Additional analyses were performed, employing 12 more melanoma cell lines. Inhibition of ATF2 expression revealed that four of the twelve exhibited increase in MITF expression, while six of the twelve decreased MITF expression. Two of the 12 lines did not exhibit change in MITF expression following ATF2 KD (Table 4). Collectively, out of 18 melanoma lines it was found that 8 (44%) retained similar negative regulation of MITF by ATF2 as observed in the melanocytes. However, another 8 (44%) exhibited positive regulation of MITF by ATF2, pointing to a transcriptional switch that occurred during the course of melanocyte transformation. MITF was not affected by altered ATF2 expression in 2/18 cell lines (Table 4). Overall, in about 50% of the melanoma cell lines, ATF2 elicits negative regulation of MITF, similar to findings with human and mouse melanocytes.

### EXAMPLE 7

### ATF2 Regulation of MITF is Mediated by Discrete Promoter Elements

MITF transcription is regulated by complex positive and negative cues. For instance, while CREB and SOX10 positively regulate MITF, BRN2, and FOXD3 have been shown to down-regulate MITF expression. Hence melanocytes and representative melanoma lines were utilized to assess mechanisms underlying positive or negative regulation of MITF. Infection of the human melanocyte line Hermes 3A with shATF2 effectively inhibited ATF2 expression, up-regulated Mitf transcription, and increased transcription of SOX10 and FOXD3 (from 7- to 10-fold, respectively) and to a lesser extent of Pax3 and Brn2 (from 1.5- to 2-fold, respectively) (FIG. 7A). Similarly, inhibition of ATF2 transcription in human melanoma 1361 cells increased SOX10 and FOXD3 transcription, albeit, to a lesser degree (3- and 1.5-fold, respectively) compared with human melanocytes. Neither BRN2 nor PAX3 transcription was elevated in melanoma cells in which ATF2 expression was inhibited. These observations suggest a role for ATF2 in FOXD3- and SOX10-mediated regulation of MITF transcription in melanocytes and melanoma cells.

To assess the possible role of FOXD3 in regulation of MITF, FOXD3 expression was inhibited in melanocytes expressing control shRNA and shATF2. Inhibition of FOXD3 expression increased SOX10 transcription and protein expression, albeit to lower levels compared to inhibition of ATF2 expression. Concomitant increase of MITF RNA and protein levels was also lower, compared with that seen upon inhibition of ATF2 expression. Notably, inhibition of both ATF2 and FOXD3 resulted in additive increase of SOX10 and MITF. These data suggest that FOXD3 may also contribute to negative regulation of MITF in melanocytes, independent of ATF2. Because inhibition of FOXD3 elicited a less pronounced effect compared with ATF2, and given that the effect appeared ATF2-independent and did not appear to mediate similar changes in human melanoma cells (data not shown), an assessment of direct mechanisms underlying ATF2 effect on MITF transcription was made.

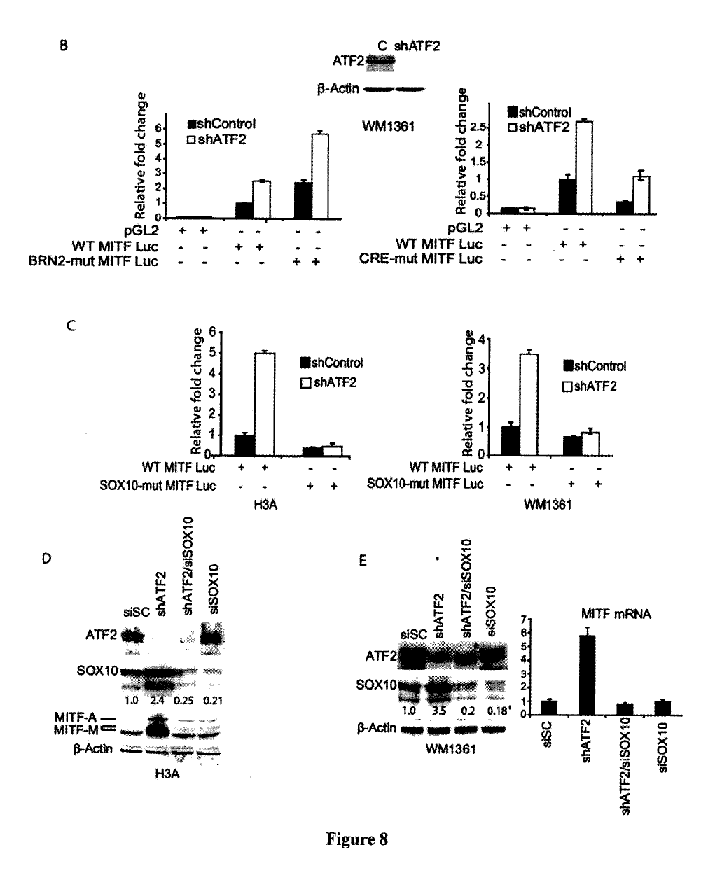

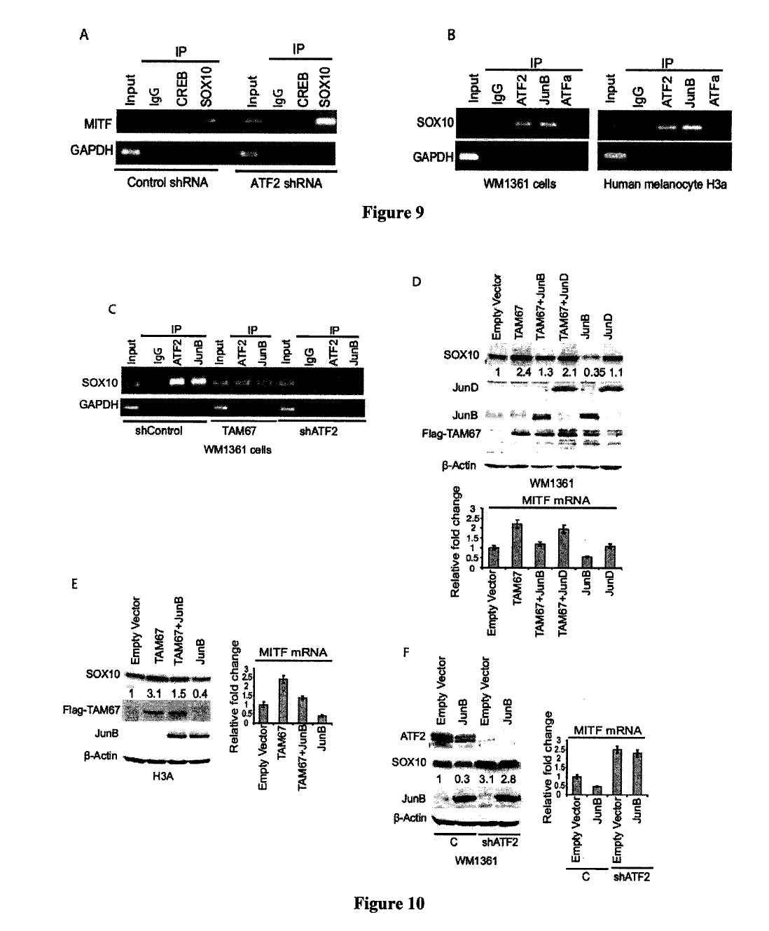

To this end, MITF promoter sequences for ATF2/CRE elements (Cyclic AMP response element), which can be targeted by ATF2, as well as sequences recognized by BRN2 and SOX10 using a luciferase reporter construct (MITF-Luc) were first analyzed. Using either a wild-type (WT) construct or one in which the BRN2 site was mutated, increased luciferase activity following inhibition of ATF2 transcription in WM1361 melanoma (FIG. 8B), as well as in LU1205 and WM35 melanoma cells (not shown) was observed. The relative increase in luciferase activity following ATF2 inhibition was equivalent in both constructs, suggesting that an ATF2 effect is not mediated by BRN2 (FIG. 8B, left panel). Similarly, MITF transcriptional activities were altered to a similar degree following inactivation of the CRE element (FIG. 8B, right panel), suggesting that ATF2 down-regulation of the MITF promoter is indirect. Therefore, an assessment was made as to whether SOX10, which positively regulates MITF and whose transcription markedly increases in melanocytes and melanoma cells in which ATF2 expression is inhibited (FIG. 7A), may mediate ATF2 effect on MITF transcription. Analysis of a MITF-Luc construct harboring a mutant SOX10 binding site revealed that ATF2 inhibition no longer elicited increased MITF transcription in human melanocytes or in melanoma cells (FIG. 8C). In agreement with the forgoing observations, inhibition of SOX10 expression by corresponding siRNA attenuated the increase in MITF transcription seen in shATF2-expressing human melanocytes (FIG. 8D) or melanoma cells (FIG. 8E). These results suggest that ATF2 regulation of MITF transcription is mediated by SOX10. Chromatin immunoprecipitation (ChIP) assays confirmed increased binding of SOX10 to the MITF promoter in melanoma cells expressing shATF2 (FIG. 9A).

A putative response element for AP1 (which can serve as an ATF2 response element through ATF2 heterodimerization with JUN family members) has been identified in upstream regions of the Sox10 promoter. Potential ATF2 binding to this element by ChIP was examined and it was found that endogenous ATF2, but not ATFa, binds to that API sequence (−4797-4791) in both human melanocytes and melanoma cells (FIG. 9B). ATF2 heterodimeric partner was next identified, which could mediate negative regulation of SOX10 transcription. Among members of the JUN family implicated in transcriptional silencing is JunB. Thus, an initiative to determine if JunB functions as an ATF2 heterodimerization partner to regulate SOX10 transcription through the API site was undertaken. ChIP analysis confirmed that JunB binds to the API site found in SOX10 promoter sequences (FIG. 10C). To confirm a possible role for JunB in regulating MITF transcription the question of whether expression of TAM67, a negative regulator of Jun family members, could attenuate the binding and transcriptional activities elicited by JunB was addressed. Expression of TAM67 indeed reduced the degree of ATF2 and JunB binding to the API site on SOX10 promoter. Further, KD of ATF2 expression abolished binding of both ATF2 and JunB to the API site on the SOX10 promoter (FIG. 10C). These data confirm the presence of ATF2-JunB complex on Sox10 promoter and suggest that ATF2 recruits JunB for binding to the API site on SOX10 promoter. To assess the role of JunB on SOX10 transcription changes in Sox10 expression at the protein and RNA levels were monitored. Expression of TAM67 caused increased expression of SOX10 in both human melanoma (approximately 2-fold; FIG. 10d) and melanocytes (approximately 3-fold; FIG. 10E), indicating some relief of JunB inhibition. Co-expression of TAM67 with JunB attenuated this increase, reducing the level of Sox10 expression to basal levels (FIGS. 10D and 10E). Over-expression of JunB, but not JunD, effectively inhibited Sox10 expression in both the melanoma and melanocytes cells (FIG. 10D and 10E). These data suggest that JunB mediates inhibition of Sox10 expression. To further reveal the role of ATF2 in this inhibition, the effect of JunB on Sox10 expression in cells expressing control shRNA or shATF2 was assessed. While ectopic expression of JunB reduced the expression of Sox10 in control shRNA-expressing cells, such decrease was no longer seen in cells expressing shATF2 (FIG. 10F). Collectively, these findings suggest that ATF2, in concert with JunB, is responsible for inhibition of Sox10 expression.

The effect of ATF2 on SOX10 and MITF expression in 12 additional human melanoma cell lines was next assessed. In all cases cells were infected with shATF2 and changes in SOX10 and MITF were monitored at the level of RNA. Notably, about four of twelve melanoma lines revealed increase in both SOX10 and MITF expression upon KD of ATF2 (Table 4). In contrast, six of twelve melanoma lines revealed decrease in MITF expression, of which 5 also shown decrease in SOX10 expression, pointing to positive regulation of SOX10 and MITF in these melanoma cells. In two out of the 12 melanoma lines, ATF2 affected SOX10 but not MITF transcription. Overall, the cohort of 18 melanoma lines revealed that about 50% of the melanomas retained negative regulation of MITF by ATF2, as seen in the melanocytes (primary and cell lines) (Table 4).

To further assess whether ATF2 regulation of MITF is Sox10-dependent in melanocytes and melanoma cells, SOX10 was co-expressed in shATF2-expressing cells. As seen in earlier analyses, inhibition of ATF2 expression caused increase in MITF transcription in the human melanocytes and 4 melanoma cell lines, (WM1361, WM793, LU1205, WM35). Notably, the melanocytes and two of four melanoma cell lines revealed ATF2 effect on MITF expression is Sox10-dependent (WM1361, WM793). Two of the four melanoma cell lines did not reveal increased SOX10 expression, although they retained increased MITF expression, upon inhibition of ATF2 (Lu1205, WM35). These findings confirm that, while in melanocytes expression of SOX10 and M1TF is negatively regulated by ATF2, this mechanism is conserved in approximately half of melanomas surveyed.

Along these lines, the two melanoma lines (MeWo and 501Mel) that exhibit positive regulation of MITF by ATF2 also exhibited positive regulation of SOX10 by ATF2. Inhibition of ATF2 expression reduced SOX10 and MITF RNA and protein levels. In order to determine whether JunB lost its ability to elicit negative regulation of SOX10 and MITF in melanoma cells where ATF2 no longer inhibited SOX10 or MITF expression, those cell lines were transfected with TAM67 and JunB alone and in combination. In these cells, whereas TAM67 effectively attenuated Sox10 and MITF expression, JunB did not alter expression of these genes, suggesting that positive regulation of MITF and SOX10 by ATF2 depends on other members of the Jun family of transcription factors. Conversely, TAM67 or JunB had no effect on melanoma cells in which ATF2 inhibits MITF independently of SOX10, suggesting that in these cases, ATF2 likely cooperates with transcription factors other than JunB to elicit negative regulation of SOX10 and MITF. Consistent with this observation, ChIP assay confirmed ATF2 and CREB, but not JunB, binding to the Sox10 promoter in these cells. These findings suggest that changes in ATF2 heterodimeric partner (from JunB to CREB) are likely to cause the switch from negative to positive regulation of SOX10, and in turn, MITF (see below). The possibility that altered expression of JunB may account for ATF2 positive or negative regulation of Sox10 and MITF was excluded, as no clear correlation between JunB expression and the ability of ATF2 to elicit negative regulation of Sox10/MITF was observed.

Among response elements potentially required to up-regulate MITF transcription is the CRE element, which is implicated in CREB-mediated upregulation of MITF transcription. Although transcriptional activity from a CRE mutant MITF promoter was lower compared to the WT promoter (30%), it was no longer responsive to inhibition of ATF2 expression in the MeWo cells. Pull-down assays using biotin-tagged MITF promoter sequences harboring the CRE identified ATF2 and CREB as CRE-bound proteins in MeWo melanoma cells. In agreement, ChIP analysis confirmed occupancy of the CRE site on MITF promoter by ATF2. These findings are consistent with the fact that ATF2 heterodimerizes with CREB and with reports that p38/MAPK14 (which phosphorylates ATF2) plays an important role in MITF transcription dependent on the CRE site. These results establish that ATF2-dependent activation of MITF transcription in these melanoma cells is mediated through the CRE site, likely in cooperation with CREB. Notably, MeWo and 501Mel lines are known to express high MITF levels compared to other melanoma lines, suggesting these cells harbor distinct mechanisms that preclude negative regulation of MITF by ATF2.

### EXAMPLE 8

### Inhibition of MITF Expression Rescues Focus Formation on Soft Agar in shATF2-Expressing Melanocytes

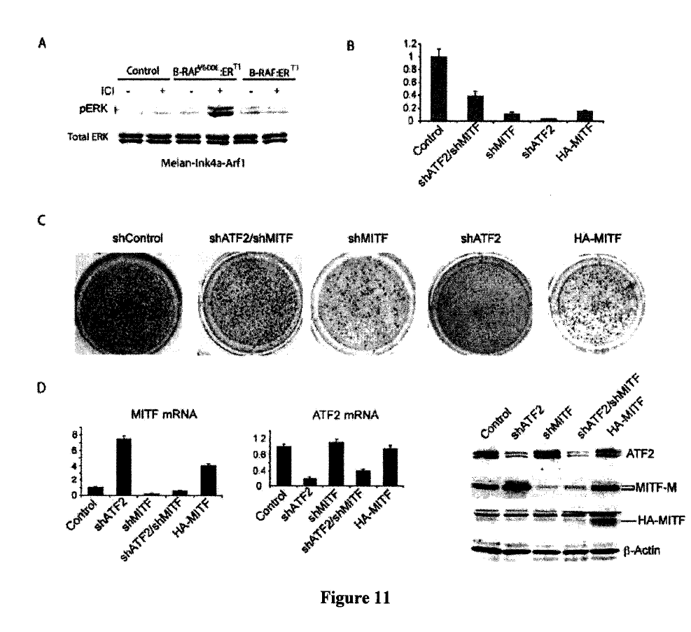

To determine whether the contribution of ATF2 to melanocyte transformation and development is MITF-dependent, melanocytes' ability to grow and form colonies in soft agar was assessed as this is indicative of their transformed potential. Expression of mutant BRAF^{V600E} in immortal melanocytes is reportedly sufficient for growth on soft agar. Thus melan-lnk4a-Arfl melanocytes were infected with mutant BRAF (FIG. 11A) and their ability to form colonies in soft agar was confirmed. Mutant BRAF expression effectively caused formation of about 1000 colonies per 5000 cells (FIGS. 11B and 11C). In contrast, melanocytes infected with BRAF^{600E} and with shATF2 formed on average about 20 colonies, indicative of loss of tumorigenicity (FIGS. 11B, 11C, and 11D) and consistent with an initial observation that the number of melanoma tumors significantly decreases in the absence of transcriptionally functional ATF2 (Tables 1 and 2). To determine the importance of MITF at this early stage of melanocyte transformation, MITF expression was inhibited (using shRNA) in melanocytes expressing mutant BRAF alone or mutant BRAF+shATF2.

Significantly, inhibition of MITF expression decreased the number of BRAF-induced foci (from 1000 to about 100 per well). Over-expression of MITF in BRAF-expressing melanocytes also inhibited focus formation, to a degree similar to that seen following inhibition of MITF expression (FIGS. 11B, 11C, and 11D). This observation implies that effective inhibition or over-expression of MITF attenuates melanocyte transformation, consistent with previous reports. Remarkably, inhibition of MITF expression in melanocytes expressing both mutant BRAF and shATF2 rescued, at least partially, melanocytes' ability to form foci on soft agar (400 compared with 20 seen in shATF2 cells; FIGS. 11B, 11C, and 11D). These findings suggest that inhibition of MITF expression in melanocytes lacking ATF2 expression can promote transformation. That MITF inhibition in melanocytes expressing ATF2 WT can attenuate their ability to form foci on soft agar is attributable to the relative expression of MITF RNA and protein in each condition (FIG. 11D). MITF expression levels in ATF2 KD cells increased 7.5-fold compared with control BRAF-expressing melanocytes. Inhibition of MITF expression in ATF2 KD melanocytes reduced MITF expression 2.5-fold relative to controls, whereas MITF KD alone resulted in lower MITF expression (5-fold; FIG. 11D). Thus, complete abrogation of MITF expression attenuates melanocyte transformation, whereas low to moderate levels of MITF expression are sufficient to promote growth on soft agar. Higher MITF expression levels, as seen in ATF2 KD cells, result in a total loss of melanocytes' ability to form foci on soft agar. These findings are consistent with the proposed rheostat model in which medium levels of MITF are optimal for growth and melanoma development and in agreement with previous observations in a mouse melanoma model.

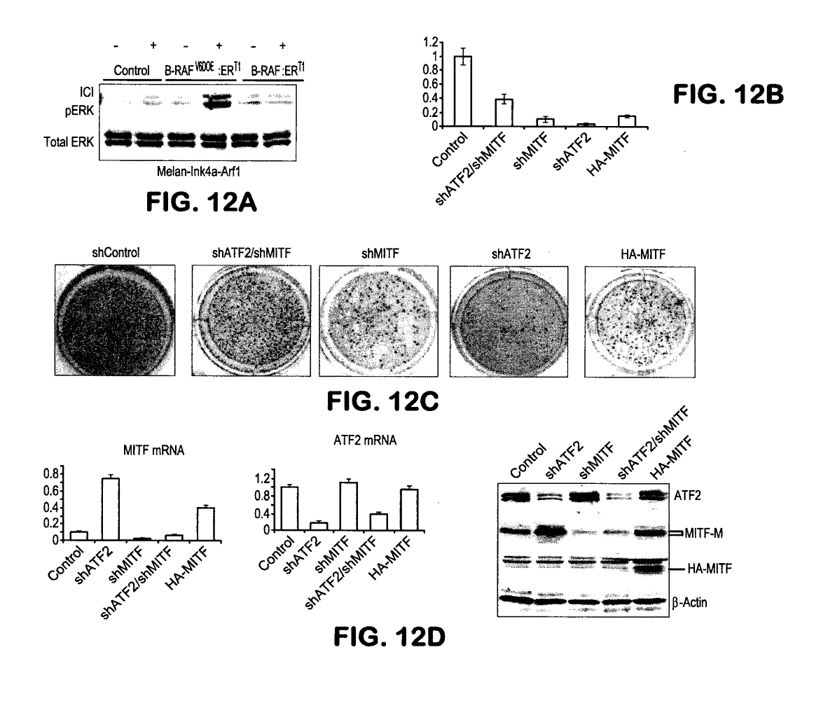

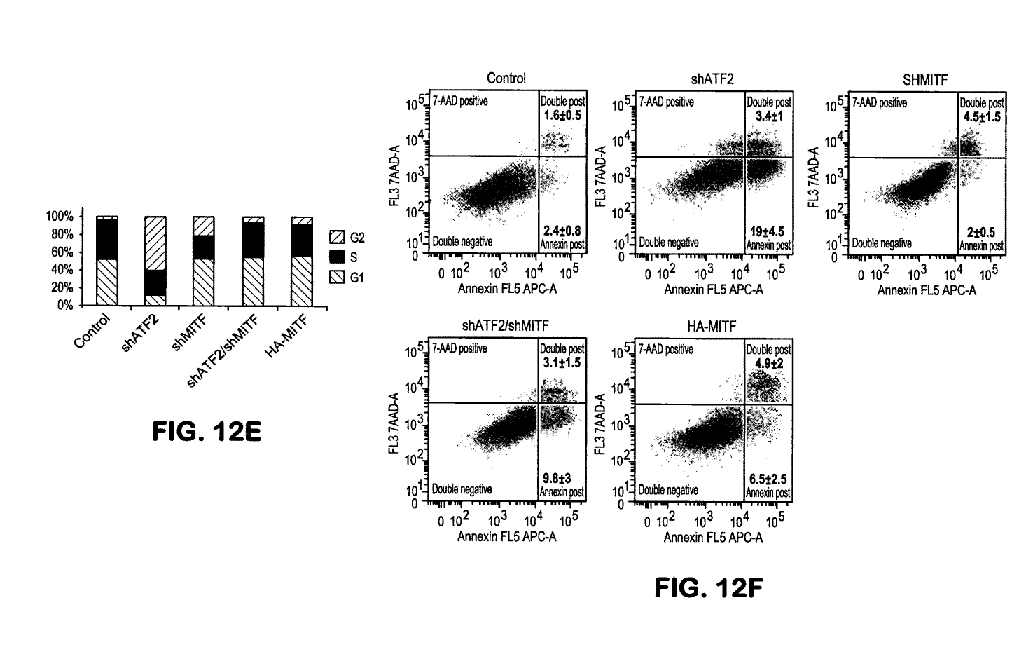

Whether inhibition of melanocyte growth on soft agar by altered ATF2 and/or MITF expression can be attributed to decreased proliferation or increased apoptosis was next addressed. Inhibition of ATF2 expression caused notable accumulation of cells in G2 (60%), with significant cell death induction (22%) compared to controls (4%), (FIGS. 12E and 12F). Interestingly, such altered cell cycle distribution and cell death rate were associated with a significant increase in MITF protein levels (FIG. 11D). In contrast, inhibition of MITF expression did not significantly induce cell death (6.5%) but resulted in fewer cells in G2/M-phase and more cells in GI, compared with inhibition of ATF2 alone. These observations suggest that MITF inhibition is sufficient to reduce the rate of cell cycle progression through G2/M phase and that inhibited growth of BRAF^{600E}-expressing melanocytes on soft agar may be attributed to abrogation of distinct cell cycle-regulatory mechanisms. Combined inhibition of ATF2 and MITF restored cell cycle distribution to that seen in control melanocytes, and reduced cell death from 22.4% to 12.9%. Of interest, MITF over-expression promoted a similar degree of cell death (11.4%) without altering cell cycle distribution, similar to combined inhibition of ATF2 and MITF (FIGS. 12E and 12F). Together, these observations suggest that simultaneous inhibition of ATF2 and MITF averts cell cycle abrogation induced when expression of either of these factors is perturbed individually, further substantiating regulation of MITF by ATF2.

### EXAMPLE 9

### Low Nuclear MITF Expression in Melanoma Tumors that Exhibit Strong Nuclear ATF2 Expression is Associated with Poor Prognosis

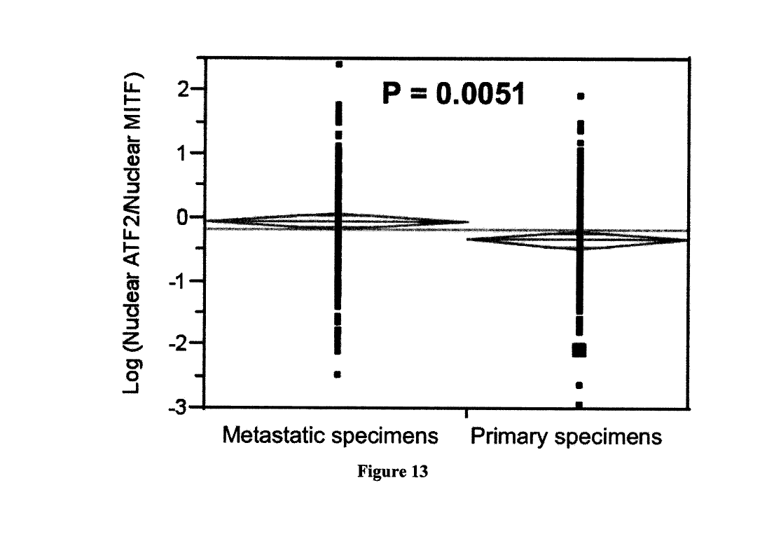

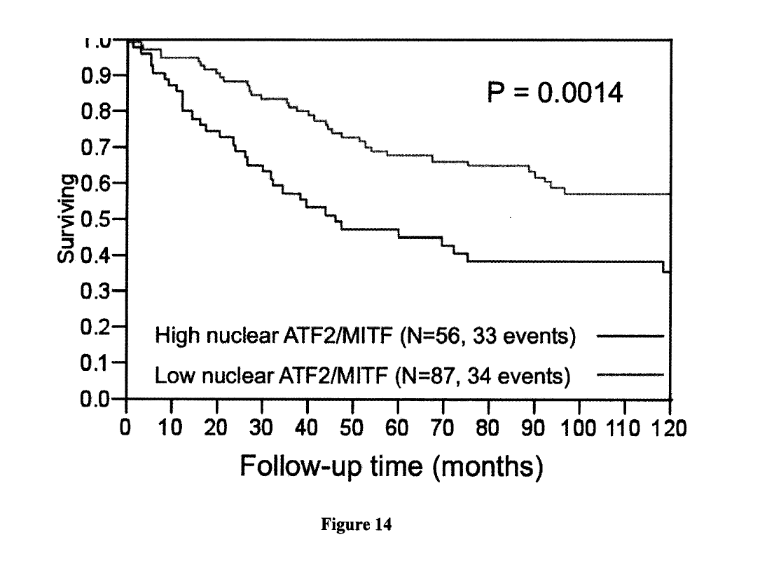

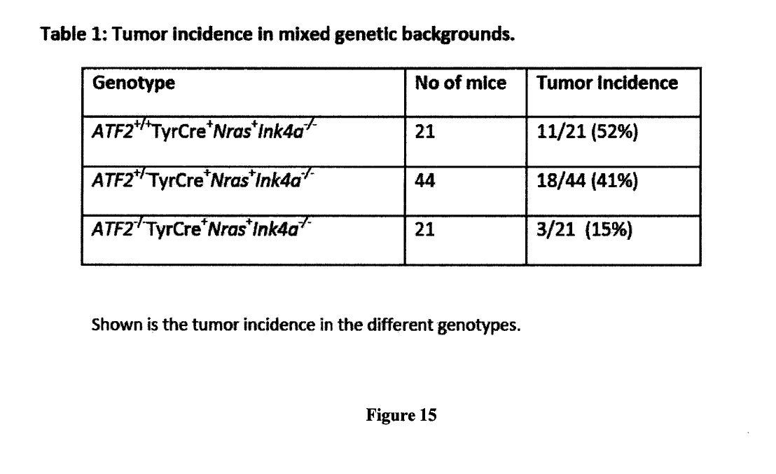

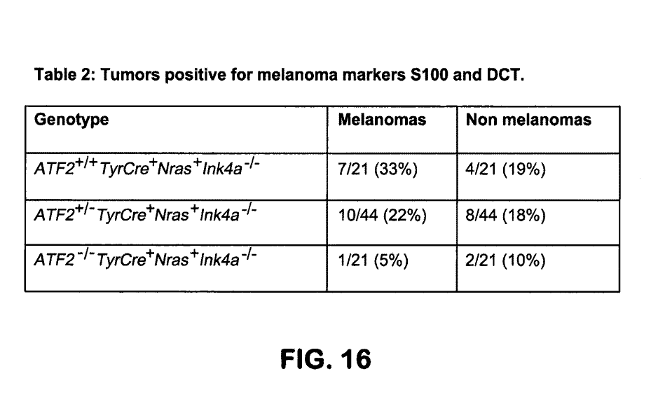

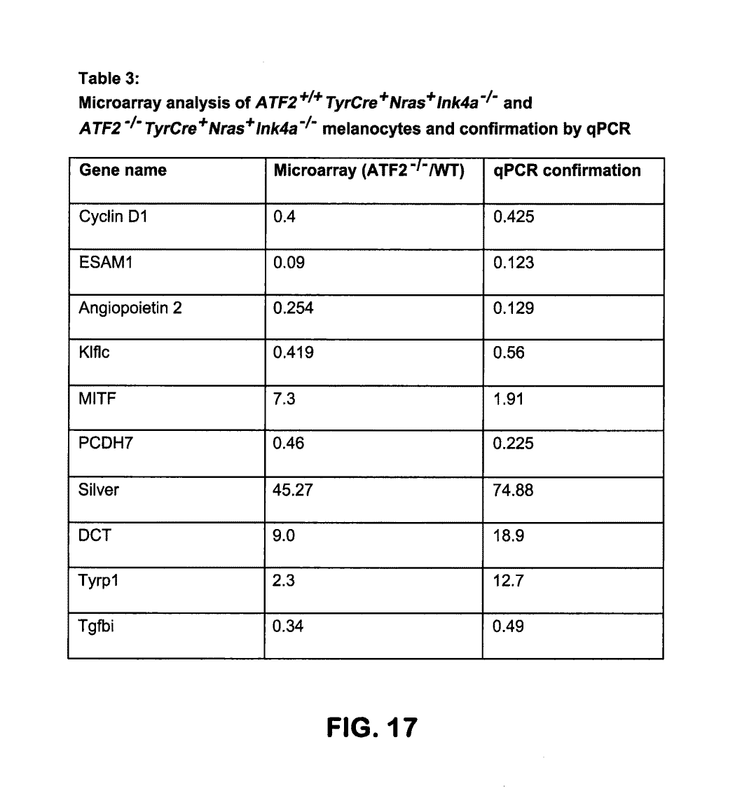

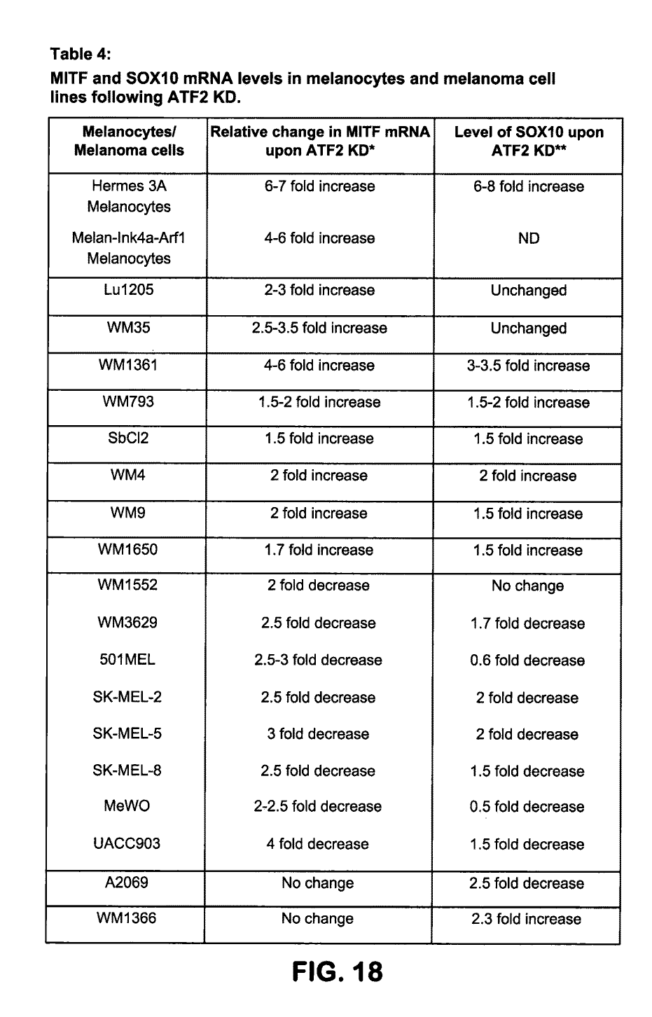

The availability of a melanoma TMA, consisting of over 500 melanoma samples and in which expression of both ATF2 and MITF in the same tumors had been measured, allowed for the assessment of possible associations between ATF2 and MITF and their correlation with survival and other clinical and pathological factors. Previous studies revealed that ATF2 sub-cellular localization in tumors is significantly correlated with prognosis: nuclear localization, reflecting constitutively active ATF2, was associated with metastasizing tumors and poor outcome. Here, immunofluorescent staining of TMAs for MITF and ATF2 was quantified employing an automated, quantitative (AQUA) method. To normalize ATF2 and MITF levels, expression of each of the two proteins in individual patients was divided by the median expression level of the respective protein in all patients, and the nuclear ATF2/MITF ratio was calculated and log-transformed. By ANOVA analysis, the ratio was higher in metastatic than in primary specimens (t value=2.823, P=0.0051), as shown in FIG. 13. No association was found between nuclear ATF2/MITF ratio and disease-specific survival among patients with metastatic melanoma (not shown). Significantly, a high nuclear ATF2/MITF ratio in primary melanoma specimens was associated with decreased 10-year disease-specific survival (P=0.0014; FIG. 14). On Cox multivariate analysis, this association with survival was independent of patient age, Breslow thickness or the presence or absence of ulceration (data not shown). Nuclear ATF2 alone in primary specimens was associated with poor survival, but to a lesser degree than the ratio of nuclear ATF2/MITF (P=0.0118 for ATF2 as a single discriminator versus P=0.0014 for the ratio of nuclear ATF2/MITF). Nuclear MITF as a single discriminator was not a significant predictor of survival (P=0.185), as was reported previously using immunohistochemistry. These observations suggest that active (nuclear) ATF2 in melanoma can suppress MITF expression, and that this phenomenon is associated with poor prognosis.

### EXAMPLE 10

### Materials and Methods

Ethics statement. Research involving human participants has been approved by the institutional review board at Yale University (where the TMA was prepared and analyzed). All animal work has been conducted according to relevant national and international guidelines in accordance with recommendations of the Weatherall report and approved by the IACUC committee at SBMRI.

Animal treatment and tumor induction protocols. Mice bearing a conditional allele for mutant ATF2 in which the DNA binding domain and part of the leucine zipper domain were deleted, were generated as previously described Genes Dev., 2007, 21: 2069-2082 and Proc. Natl. Acad. Sci., 2008, 105:1674-1679. The Cre-loxP system for disruption of the ATF2 gene in melanocytes was utilized to study the function of ATF2 in melanocytes. The Tyr::Cre^{ER}::/Atf2^{md} mice and their littermate controls (WT) were of FVB/129P2/OlaHsd (TyrCre^{ERT} mice were FVB, ATF2^{fl/fl} were 129P2/OlaHsd) and N-Ras/lnk4a^{-/-} mice were C57BI/6/129SvJ. For melanoma studies Tyr::Cre^{ER}::Nras^{Q61K}::Ink4a^{-/-} mice (developed at HMS by LC) were used following their cross with the Tyr::Cre^{ER}::Atf2^{md}.

Immunohistochemistry—Skin specimens were fixed in neutral buffered formalin solution and processed for paraffin embedding. Skin sections (5 pm in thickness) were prepared and deparaffinized using xylene. For MITF, DCT and S100 immunostaining, tissue sections were incubated in DAKO antigen retrieval solution, for 20 minutes in a boiling bath, followed by treatment with 3% hydrogen peroxide for 20 minutes. Antibodies against MITF (1:100 from Sigma), DCT (1:500, kind gift from Dr. Vincent Hearing) and 5100 (1:100, DAKOCytomation; Carpinteria, Calif.) were allowed to react with tissue sections at 4° C. overnight. Biotinylated anti-rabbit IgG was allowed to react for 30 minutes at ambient temperature and diaminobenzidineor Nova Red were used for the color reaction while hematoxylin was used for counterstaining. The control sections were treated with normal mouse serum or normal rabbit serum instead of each antibody.

Cell culture—Immortalized human melanocytes Hermes 3A, which exhibit hTERT (puro) and CDK4 (neo) expression were grown in RPMI 1640 medium containing Fetal Bovine Serum (FBS, 10%), 12-0-tetradecanoyl-phorboi-13-acetate (TPA, 200 nM, Sigma, St. Louis, Mo.), Cholera toxin (200 pM, Sigma), human stem cell factor (10 ng/ml, R&D systems, Minneapolis, Minn.), and endothelin 1 (10 nM, Bachem Bioscience Inc., Torrance, Calif.). Primary human melanocytes (NEM-LP; Invitrogen) were grown in medium 254 and HMGS (Cascade Biologies). Mouse melanocytes (melan-lnk4a-Arf1) were grown as for immortalized human melanocytes excluding human stem cell factor and endothelin. Melanoma cell lines were grown in DMEM medium supplemented with 10% FBS and penicillin/streptomycin (P/S; Cellgro). Melanoma cell lines used in this study include LU1205, WM793, 501MEL, WM35, WM1361 MeWO (kind gift from Meenhard Herlyn) maintained in DMEM medium supplemented with 10% FBS and Penicillin/Streptomycin. Primary melanocytes cultures were prepared from mice carrying the Atf2 WT or mutant genotypes and N-Ras/lnk4a^{-/-} as follows. Dorsal-lateral skin was removed from one day-old pups, disinfected with 70% ethanol for 1 minute and then wash at least twice with sterile PBS. The skin was submerged in 1× Trypsin/EDTA overnight at 4° C. and next day, the skin was placed in a Petri dish with mouse melanocyte culture medium (described below). The epidermis and sheared tissue was removed and discarded with forceps. The tissue was transferred to 15 ml centrifuge tubes and vortexed vigorously until solution becomes cloudy (1-2 minutes). The cell suspension was transferred to tissue culture flasks. After 3 days, melanocyte growth medium containing 0.8 jig/ml geneticin (Sigma-Aldrich) was added to eliminate contaminating fibroblasts (melanocytes are resistant to such treatment). Geneticin-containing medium was removed and replaced with fresh media after 1 day. Media was changed twice a week. Primary mouse melanocytes were grown in F-12 media (invitrogen) containing 20% L-15 media (Invitrogen), 4% of FBS and Horse serum (Invitrogen), Penicillin (100 units) and streptomycin (50 μg) antibiotics, db-cAMP (40 μM, Sigma-Aldrich), 12-0-tetradecanoyl-phorbol-13-acetate (TPA, 50 ng/ml, Sigma-Aldrich), alpha-Melanocyte stimulating hormone (a-MSH, 80 nM, Sigma-Aldrich), Fungizone (2.5 μg/ml, Sigma-Aldrich) and melanocyte growth supplement (Invitrogen). Primary melanocytes were treated with 4-OHT (10 μM) for 8 h followed by addition of doxycycline (2 μg/ml) for 24 hours to inactivate ATF2 and induce expression of N-Ras.

Constructs—ATF2-specific shRNA clones were obtained from Open Biosystems (catalog number: RHS4533). Five different shRNA were obtained and tested for their efficiency of KD. Clone TRCN0000013714 was more efficient in inhibiting ATF2 in human cell lines while clone TRCN0000013713 was more efficient for knocking down mouse ATF2. For subsequent experiments the respective shATF2 clone was used depending on human or mouse cell lines. Three different clones were also tested for KD of ATF2 to rule out any off-target effect (data not shown). siRNA control (catalog number: 4611) and three Sox10-specific siRNA oligonucleotides were obtained from Ambion (catalog number: 4392420). Four FOXD3 specific siRNA were obtained from Dharmacon (catalog numbers: J-009152-06, -07, -08, and -09). These siRNAs were pooled together in an equimolar ratio for transient transfection. An MITF specific shRNA, and MITF promoter luciferase constructs (WT and mutant CRE-Luc constructs) were obtained from Dr. David Fisher (J. Biol. Chem., 2003, 278: 45224-45230). pGL3 vectors containing wild-type and BRN2-site-mutated MITF promoters were obtained from Dr. Colin Goding (Cancer Res., 2008, 68: 7788-7794). pGL3 vectors containing wild-type and Sox10-site-mutated MITF promoters were obtained from Dr. Michel Goossens (Hum. Mol. Genet., 2000, 9: 1907-1917). Retroviral vectors encoding a fusion protein consisting of full length human BRAF and BRAF^{V600E} linked to the TI form of the human estrogen receptor hormone-binding domain were generously provided by Dr. Martin McMahon (Pigment Cell Melanoma Res., 2008, 21: 534-544). SOX10 expression vector obtained from Dr. Alexey Terskikh, RSV-JunB, RSV-JunD were obtained from Dr. Michael Karin and pBabe-Flag-TAM67 from Dr. Michael Birrer.

Antibodies and Immunoblotting—Antibodies against SOX10 and CREB (sc-1734 and sc-186 respectively) were from Santa Cruz Biotechnologies; antibodies against ATF2, pERK and ERK (catalog numbers: 9226, 4337 and 4695, respectively) were obtained from Ceil Signaling; antibodies against MITF (C5) were purchased from Cell Lab vision. Protein extract (40-60 μg) preparation and western blot analysis were done as described previously in Cancer Cell, 2007, 11: 447-460. Specific bands were detected using fluorescent-labeled secondary antibodies (Invitrogen, Carlsbad, Calif.) and analyzed using an Odyssey Infrared Scanner (Li-COR Biosciences). p-Actin antibody was used for monitoring loading.

Immunofluorescence—Human melanoma and melanocytes were grown in coverslips, fixed (4% paraformaldehyde and 2% sucrose in 1× PBS), and then permeabilized and blocked (0.4% Triton X-100 and 2% BSA in 1× PBS) at ambient temperature. The cells were then washed (0.2% Triton X-100 and 0.2% BSA in 1× PBS) and incubated overnight at 4° C. with monoclonal anti-rabbit antibody against ATF2 (20F 1, 1:100), followed by five washes and then subsequent incubation at ambient temperature for 2 hours with anti-rabbit IgG (Invitrogen, 1:300) and Phalloidin (Molecular Probes, 1:1000). DNA was counterstained with 4,6-diamidino-2-phenylindole (DAPI; Vector Laboratories) containing mounting medium.

Analysis of Skin samples—Skin samples were collected from the backs of mice and immediately fixed with Z-fix, processed, and embedded in paraffin. Paraffin sections were routinely stained by H&E. Dewaxed tissue sections (4.0-5.0 μm) were immunostained using rabbit polyclonal antibodies to MITF (Sigma-Aldrich), S100 (S100B; DAKOCytomation; Carpinteria, Calif.), and DCT (aPEPS, kindly provided by Dr. Vincent Hearing). Application of the primary antibody was followed by incubation with goat anti-rabbit polymer-based EnVision-HRP-enzyme conjugate (DakoCytomation). DAB (DakoCytomation) or SG-Vector (Vector Lab, Inc.; Burlingame, Calif.) chromogens were applied, yielding brown (DAB) and black (SG) colors, respectively.

Quantitative Analysis of Immunostaining—Quantitative analysis was performed as described previously in J. Histochem. Cytochem., 2009, 57: 649-663. Briefly, all slides were scanned at an absolute magnification of 400× [resolution of 0.25 μm/pixel (100,000 pix/in.)] using the Aperio ScanScope CS system (Aperio Technologies; Vista, Calif.). The acquired digital images representing whole tissue sections were analyzed applying the Spectrum Analysis algorithm package and ImageScope analysis software (version 9; Aperio Technologies, Inc.) to quantify IHC and histochemical stainings. These algorithms make use of a color deconvolution method (Anal. Quant. Cytol. Histol., 2001, 23: 291-299) to separate stains. Algorithm parameters were set to achieve concordance with manual scoring on s number of high-power fields, including intensity thresholds for positivity and parameters that control cell segmentation using the nuclear algorithm.

Microarray analysis—Primary melanocytes were treated with 4-OHT and Doxycycline before isolation of total RNA. 500 ng of total RNA was used for synthesis of biotin-labeled cRNA using an RNA amplification kit (Ambion). The biotinylated cRNA is labeled by incubation with streptavidin-Cy3 to generate probe for hybridization with the Mouse-6 Expression BeadChip (Illumina MOUSE-6_V1_{—}1_{—}11234304_A) that represents 46.6 K mouse gene transcripts. BeadChips were analyzed using the manufacturer's BeadArray Reader and collected primary data using the supplied Scanner software. Data analysis was done as follows. First, expression intensities were calculated for each gene probed on the array for all hybridizations using illumina's BeadStudio 3.0 software. Second, intensity values were quality controlled and normalized: quality control was carried out by using the BeadStudio detection P-value set to <0.01 as a cutoff. This removed genes not detected in the arrays. All the arrays were then normalized using the cubic spline routine from the BeadStudio 3.0 software. This procedure accounted for any variation in hybridization intensity between the individual arrays. Finally, these normalized data were analyzed for differentially expressed genes. The groups of 2 biological and 2 technical replicates were described to the BeadStudio 3.0 software and significantly differentially expressed genes were determined on the basis of the difference changes in expression level (Illumina DiffScore>60 or DiffScore<−60) and expression difference p-value<0.01. Microarray data are available under accession number GSE23860.

ShRNA infection and RNA interference—Human embryonic kidney 293T cells were transfected with corresponding retro-or lentiviral shRNA constructs (10 μg), Gag-pol (5 μg) and ENV expression vectors (10 μg) by calcium phosphate transfection into 10 cm plates and supernatant was collected after 48 hours to obtain viral particles. Two million melanocytes and melanoma cells in 10 cm plates were infected with 5 ml of viral supernatant along with 5 ml of medium in the presence of 8 μg/ml polybrene. The virus was replaced with fresh media after 8 hours of infection. After two days, puromycin (1.5 μg/ml} was used to select cells for 3 days. For human and mouse melanocytes the media was changed to DMEM containing 10% FBS 24 hours prior to harvesting cells. 50 nM duplexes of scrambled and SOX10- or FOXD3-specific siRNA were transfected into human melanocytes and WM1361 melanoma cells (2 million cells per transfection) by Nucleofection using Amaxa reagents (NHEM-Neo Nucleofector and Solution R, respectively) for SOX10 or FOXD3 knock down. Over 90% of the cells transduced were able to resist drug selection, indicating efficient infection of the respective genes. GFP was also used to monitor efficiency of infection, confirming >90% GFP expression by fluorescence microscopy.

Real-time quantitative reverse transcription-PCR (RT-PCR)—Quantitative PCR was performed as described above. Total RNA was isolated using an RNeasy mini kit (Sigma, St. Louis, Mo.) and reverse transcribed using a high cDNA capacity reverse transcription kit (Applied Biosystems, Foster City, Calif.) following the manufacturer's instructions.

Specific primers (Valuegene, San Diego, Calif.) used for PCR were as follows:

Human ATF2,
forward: tgtggccagcgttttaccaa,
reverse: tgatgtgggctgtgcagttt.,
human MITF,
forward: aaaccccaccaagtaccaca,
reverse: acatggcaagctcaggac,
human SOX10,
forward: caagtaccagcccaggcggc,
reverse: gggtgccggtggtccaagtg.,
human FOXD3,
forward: gcgacgggctggaagag,
reverse: gctgtccgtgatggggtgcc,
human PAX3,
forward: ggaactggagcgtgcttttg,
reverse: ggcggttgctaaaccagac,
human BRN2,
forward: gaaagagcgagcgaggaga,
reverse: caggctgtagtggttagacg.,
mouse MITF,
forward: agatttgagatgctcatcccc,
reverse: gatgcgtgatgtcatactgga,
mouse TYRP1,
forward: ccctagcctatatctccctttt,
reverse: taccatcgtggggataatggc,
mouse DCT,
forward: gtcctccactcttttacagacg,
reverse: attcggttgtgaccaatgggt,
mouse Silver,
forward: tgacggtggaccctgcccat,
reverse: agctttgcgtggcccgtagc.

The reaction mixture was denatured at 95° C. for 10 minutes, followed by 40 cycles of 95° C. for 15 seconds, annealing at 60° C. for 30 seconds and extension at 72° C. for 30 seconds. Reactions were performed using the SYBR® Green^{ER} qPCR reagent (Invitrogen) and run on an MX3000P qPCR machine (Stratagene, La Jolla, Calif.). The specificity of the products was verified by melting curve analysis and agarose gels. The amount of the target transcript was related to that of a reference gene (Cyclophilin A for both human and mouse) by the Ct method. Each sample was assayed at least in triplicate and was reproduced at least three times.

Chromatin Immunoprecipitation—Chromatin immunoprecipitation was performed using the Magna-Chip (Upstate) according to the manufacturer's instructions. Control shRNA and ATF2 knocked down WM1361 cells (one 10 cm plate for each, 80% confluent) were fixed in 37% formaldehyde and sheared chromatin was immunoprecipitated and subjected to PCR for 26 cycles. The following primers corresponding to the MITF promoter, spanning the SOX10 binding site which also includes a CRE site, were used, forward: gcagtcggaagtggcag, reverse: caactcactgtcagatcaa. Antibodies against Sox10 and CREB (se-1734 and sc-186 respectively) were from Santa Cruz Biotechnologies. IgG control, and glyceraldehyde-3-phosphate dehydrogenase oligonucleotides were provided by the kit. Antibody against ATF2 (sc-6233), JunB (sc-8051), JunD (sc-74) were obtained from Santa Cruz. Antibodies against ATFa were provided generated by Nic Jones. For Sox10 promoter, the following primers spanning AP-1 binding site were used; forward: cccagtgctggcctaatagc, reverse: cacccttgatatccccaagtga.

Luciferase Assays—MeWo, WM35, WM1361, Lul205 cells in six-well plates were transiently transfected with 0.5 μg of reporter plasmid containing WT or CRE mutant, BRN2 mutant or SOX10 mutant MITF promoter and 0.1 μg of pSV-B-Galactosidase (Promega, San Luis Obispo, Calif.) using Lipofectamine 2000 reagent (InVitrogen). Human melanocytes (2 million) were transfected with 2 μg of reporter plasmid containing WT or SOX10 mutant MITF promoter and 0.3 μg of pSV-(i-Galactosidase using Amaxa reagent (NHEM-Neo nucleofector kit, Lonza) according to the manufacturer's protocol. Cell lysates were prepared from cells after 48 hours. Luciferase activity was measured using the Luciferase assay system (Promega) in a luminometer and normalized to B-galactosidase activity. The data were normalized to B-galactosidase and represent the mean and SD of assays performed in triplicate. All experiments were performed a minimum of 3 times.

Colony formation assay—Melan-lnk4a-Arf1 cells were transduced with a retroviral vector expressing BRAF^{V600E}:ER^{T1}and selected with puromycin for 3 days. These cells were treated with 200 nM of estrogen receptor antagonist ICI 182780 (ICI, Tocris Bioscience) to induce expression of BRAF^{V600E}. After one day, these cells were transduced with a lentiviral vector expressing either shATF2 or shMITF separately, or in combination. Colony formation was carried out as described by Franken et al. (Clonogenic assay of cells in vitro, 2006). Briefly, 5,000 cells were plated into each well of a 6-well plate, and cells were grown in mouse melanocyte media containing ICI and puromycin (1.5 μg/ml) for 3 weeks until colonies became visible. The colonies were stained with P-Iodonitrotetrazolium Violet (1 mg/ml Sigma, St. Louis, Mo.). This experiment was performed in triplicate and reproduced 2 times.

Mouse genotyping—Genomic DNA was isolated from tail tissue was subjected to PCR resulting in amplification of a 549 bp DNA fragment for Atf2 floxed and a 485 bp DNA fragment for wild type mice. PCR conditions included one cycle at 95° C. for 3 minutes; and 30 cycles of 94° C./30 seconds, 55° C./30 seconds and 72° C./1 minute and one cycle at 72° C. for 5 minutes. Primers used for PCR reactions were forward: caatccactgccatggcctt, reverse: tcagataaagccaagtcgaatctgg.

Avidin-Biotin DNA-protein binding assay—MeWo cells were left untreated or treated with 20 mJ/cm² of UV-B for 1 hour. The cells were lysed using lysis buffer containing 1% Triton-100 and incubated with 4 μg of biotin-labeled MITF promoter spanning the CRE site oligo (5′-gaaaaaaaagcatgacgtcaagccaggggg-3′) in the presence of poly-(dl-dC) (20 μg/ml) for 2 hours at 4° C. The oligo-bound proteins were captured using streptavidin-agarose (Invitrogen) for 1 hour incubation, followed by extensive washes with washing buffer (20 mm HEPES, 150 mm NaCI, 20% glycerol, 0.5 mm EDTA, and 1% Triton-100) and analyzed using SDS-PAGE and western blots.

BrdU, PI Labeling, Annexin V staining—To evaluate the cell cycle index of Melan-lnk4a-Arf1 cells stably over-expressing BRAF^{V600E}:ER^{T1} alone or in combination with shRNA to the genes described above, cells were plated in media containing ICI and puromycin (1.5 μg/ml) at 2×10⁶ cells per 10 cm plate over night. Cells were labeled with 10 μM of 5-bromo-2-deoxyuridine (BrdU; Sigma Chemical Co.), for an hour. Cells were then washed, fixed, and stained with anti-BrdU mAbs and propidium Iodide (BD Biosciences, San Jose, Calif.) according to the manufacturer's protocol, and analyzed on a BD FACSCanto machine. Cell cycle phase was analyzed using the Mod Fit LT v.2 program (Verity Software, Topsham, Me.). In a separate experiment the cells were stained with Annexin V-APC and 7-AAD (BD Pharmingen™, San Diego, Calif.) according to manufacturer's protocol, to enable analysis of early apoptosis and cell death.

Hypoxia treatment—Cells were treated under hypoxia (1% O₂) for indicated time points using a hypoxia chamber (In Vivo 400; Ruskin Technologies Ltd, Bridgend, UK).

UV Irradiation—Mice were treated with 4-hydroxytamoxifen (25 mg/ml in dimethylsulfoxide) by swabbing the entire body (excluding the head) on days 1 through 3 after birth. On day 4, the pups were placed under UVB light source (FL-15E; 320 nm) and exposed to 20 μW/cm² for 22 seconds. Ninety minutes after UVB treatment mice were sacrificed and entire skin was removed and processed.

TMA and AQUA staining—Tissue microarrays were constructed as previously described in Cancer Res., 2003, 63: 8103-8107. The arrays included a series of 192 sequentially collected primary melanomas and 299 metastatic melanomas. Slides were stained for automated, quantitative analysis (AQUA) for ATF2 and MITF as previously published (Nature, 2005, 436: 117-122 and J. Clin. Oncol., 2009, 27: 5772-5780). The AQUA scores for the two markers were obtained from the AQUAmine database at the world wide web tissuearray.org.

Although the invention has been described with reference to the above example, it will be understood that modifications and variations are encompassed within the spirit and scope of the invention. Accordingly, the invention is limited only by the following claims.

What is claimed is:

1. A method for the diagnosis or prognosis of melanoma in a subject comprising obtaining a nucleic acid sample from melanocytes of a subject suspected of having melanoma or at risk of having melanoma; detecting expression of activating transcription factor 2 (ATF2) and microphthalmia-associated transcription factor (MITF) in melanocytes; and determining a ratio of ATF2:MITF expression, thereby allowing diagnosis or prognosis of melanoma in the subject.

2. The method of claim 1, wherein detection of expression is a comparison of nuclear and cytoplasmic localization of ATF2 expression in melanocytes.

3. The method of claim 1, wherein an increased ratio of nuclear localization of ATF2:MITF expression in melanocytes is associated with metastatic melanoma.

4. A method of treating a melanocyte proliferative disorder in a subject comprising administering to the subject an effective amount of an agent that modulates the transcriptional activity of ATF2, thereby treating the disorder.

5. The method of claim 4, further comprising administering an agent that increases the expression of MITF.

6. The method of claim 4, further comprising administering an agent that modulates the transcriptional activity of SOX10.

7. The method of claim 4, further comprising administering an agent that modulates the activity of melanocyte pigmentation genes.

8. The method of claim 7, wherein the pigmentation genes are selected from the group consisting of Cyclin D1, ESAM1, Angiopoietin 2, Klflc, PCDH7, Silver, DCT, Tyrp1, and Tgfbi.

9. The method of claim 4, wherein the agent inhibits the transcriptional activity of ATF2.

10. The method of claim 4, wherein the disorder is melanoma.

11. The method of claim 10, wherein the disorder is metastatic melanoma.

12. The method of claim 4, further comprising administering vemurafenib (Zelboraf™).
